(19) United States
(12) Patent Application Publication
(10) Pub. No.: US 2022/0091050 A1
(43) Pub. Date: Mar. 24, 2022

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventors: Atsushi IWASHITA, Tokyo (JP); Sota TORII, Kanagawa (JP); Takeshi NODA, Kanagawa (JP); Kosuke TERUI, Kanagawa (JP); Akira TSUKUDA, Tokyo (JP); Ryuichi FUJIMOTO, Tokyo (JP)

(21) Appl. No.: 17/540,776

(22) Filed: Dec. 2, 2021

Related U.S. Application Data
(63) Continuation of application No. PCT/JP2020/022726, filed on Jun. 9, 2020.

(30) Foreign Application Priority Data
Jun. 11, 2019 (JP) 2019-109019
May 28, 2020 (JP) 2020-093673

(51) Int. Cl. G01N 23/04; G01N 23/083

(52) U.S. Cl. CPC G01N 23/04; G01N 2223/424; G01N 2223/403; G01N 23/083

(57) ABSTRACT

An image processing apparatus generates a plurality of attenuation rate images using a plurality of radiation images corresponding to a plurality of different radiation energies obtained by irradiating an object with radiation, corrects the radiation images or the attenuation rate images so as to reduce an error of an attenuation rate caused depending on at least one of a dose of the radiation, a thickness of the object, and energy of the radiation, and generates a material characteristic image by energy subtraction processing using the plurality of attenuation rate images after the correction.

### CROSS-REFERENCE TO RELATED APPLICATIONS

This application is a Continuation of International Patent Application No. PCT/JP2020/022726, filed Jun. 9, 2020, which claims the benefit of Japanese Patent Application No. 2019-109019, filed Jun. 11, 2019 and Japanese Patent Application No. 2020-093673, filed May 28, 2020, all of which are hereby incorporated by reference herein in their entirety.

### BACKGROUND OF THE INVENTION

### Field of the Invention

The present invention relates to an image processing apparatus, an image processing method, and a program and, more particularly, to a radiation imaging apparatus preferably used for still image capturing such as general imaging or moving image capturing such as fluoroscopic imaging in imaging diagnosis, an image processing apparatus in a radiation imaging system, an image processing method, and a program.

### Background Art

A radiation imaging apparatus using a flat panel detector (to be abbreviated as an FPD hereinafter) made of a semiconductor material is currently widespread as an imaging apparatus used for medical imaging diagnosis or non-destructive inspection by X-rays. Such radiation imaging apparatus is used as a digital imaging apparatus for still image capturing like general imaging or moving image capturing like fluoroscopic imaging in, for example, medical imaging diagnosis.

One of imaging methods using an FPD is energy subtraction. In energy subtraction, for example, a plurality of images of different energies are obtained by emitting X-rays generated at different tube voltages. Processing of, for example, calculating those images to separate the processing result into a bone image and a soft tissue image can be performed (PTL 1).

### CITATION LIST

### Patent Literature

PTL 1: Japanese Patent Laid-Open No. 58-221580

When estimating a bone thickness and a soft tissue thickness using energy subtraction, an error occurs between the estimated value of each thickness and its true value arises. As a result, a problem that the accuracy of a bone density measurement deteriorates or an artifact occurs when applying image processing to the estimated bone thickness or soft tissue thickness arises.

### SUMMARY OF THE INVENTION

The present invention provides a technique of reducing an error occurring when generating a material characteristic image by energy subtraction.

According to one aspect of the present invention, there is provided an image processing apparatus comprising: a generating unit configured to generate a plurality of attenuation rate images using a plurality of radiation images corresponding to a plurality of different radiation energies obtained by irradiating an object with radiation; a correcting unit configured to correct the radiation images or the attenuation rate images so as to reduce an error of an attenuation rate caused depending on at least one of a dose of the radiation, a thickness of the object, and energy of the radiation; and a processing unit configured to generate a material characteristic image by energy subtraction processing using the plurality of attenuation rate images after the correction by the correcting unit.

According to another aspect of the present invention, there is provided an image processing apparatus comprising: a correcting unit configured to correct, based on a difference between a pixel value obtained by calculation and a pixel value obtained by actual measurement, a radiation spectrum to be used for energy subtraction processing; a generating unit configured to generate a plurality of attenuation rate images using a plurality of radiation images corresponding to a plurality of different radiation energies obtained by irradiating an object with radiation; and a processing unit configured to generate a material characteristic image by energy subtraction processing using the plurality of attenuation rate images and the radiation spectrum corrected by the correcting unit.

According to another aspect of the present invention, there is provided an image processing method comprising performing energy subtraction processing (a) using a radiation spectrum obtained by performing correction based on a plurality of attenuation rates corresponding to a plurality of different radiation energies obtained by irradiating an object with radiation and a difference between a pixel value obtained by calculation and a pixel value obtained by actual measurement, or (b) using at least one attenuation rate obtained by performing correction so as to reduce an error of at least one attenuation rate caused depending on at least one of a dose of the radiation, a thickness of the object, and energy of the radiation.

According to another aspect of the present invention, there is provided an image processing method comprising performing energy subtraction processing (a) using a radiation spectrum obtained by performing correction based on a plurality of attenuation rates corresponding to a plurality of different radiation energies obtained by irradiating an object with radiation and a difference between a pixel value obtained by calculation and a pixel value obtained by actual measurement, or (b) using at least one attenuation rate obtained by performing correction so as to reduce an error of at least one attenuation rate caused depending on at least one of a dose of the radiation, a thickness of the object, and energy of the radiation.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 a block diagram showing an example of the arrangement of an X-ray imaging system according to the first embodiment;

FIG. 2 is a pixel equivalent circuit diagram of an X-ray imaging apparatus according to the first embodiment;

FIG. 3 is a timing chart of the X-ray imaging apparatus according to the first embodiment;

FIG. 4 is a timing chart of the X-ray imaging apparatus according to the first embodiment;

FIG. 5A is a view for explaining correction processing according to the first embodiment;

FIG. 5B is a block diagram of signal processing according to the first embodiment;

FIG. 5C is a block diagram of image processing according to the first embodiment;

FIG. 6 is a view showing a graph 6a representing the dose dependence of an attenuation rate, a graph 6b representing the dose dependence of a pixel value, and a graph 6c representing a correction coefficient of the dose dependence according to the first embodiment;

FIG. 7A is a view showing correction processing according to the first embodiment;

FIG. 7B is a view showing correction processing according to the first embodiment;

FIG. 8 is a view showing a graph 8a representing the thickness dependence of an attenuation rate, and a graph 8b representing a correction coefficient of the thickness dependence according to the second embodiment;

FIG. 9 is a view showing a graph 9a representing the spectrum dependence of an attenuation rate, and a graph 9b representing a correction coefficient of the thickness dependence according to the second embodiment;

FIG. 10 is a view showing correction processing according to the second embodiment;

FIG. 11 is a view for explaining an arrangement 11a for X-ray imaging, an arrangement 11b for obtaining a correction value, and X-ray imaging 11c for obtaining a correction value according to the third embodiment;

FIG. 12 is a flowchart for explaining material decomposition image obtaining processing according to the third embodiment;

FIG. 13 is a view showing an arrangement 13a for X-ray imaging and an X-ray imaging arrangement 13b for obtaining a correction value according to the fourth embodiment; and

FIG. 14 is a flowchart illustrating material decomposition image obtaining processing according to the fifth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

Note that radiation according to the present invention includes not only α-rays, β-rays, and γ-rays that are beams generated by particles (including photons) emitted by radioactive decay but also beams having equal or more energy, for example, X-rays, particle rays, and cosmic rays. Each of the following embodiments will describe apparatuses using X-rays as an example of radiation. Therefore, X-rays, an X-ray image, X-ray energy, an X-ray spectrum, an X-ray dose, an X-ray generating apparatus, an X-ray imaging apparatus, and an X-ray imaging system will be described as radiation, a radiation image, radiation energy, a radiation spectrum, a radiation dose, a radiation generating apparatus, a radiation imaging apparatus, and a radiation imaging system.

### First Embodiment

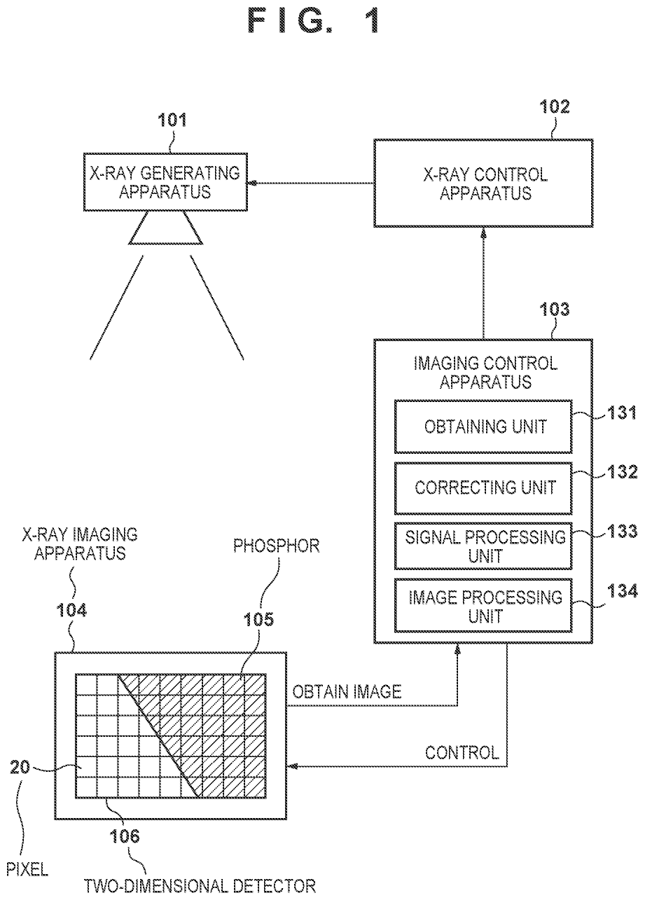

FIG. 1 is a block diagram showing an example of the arrangement of an X-ray imaging system as an example of a radiation imaging system according to the first embodiment. The X-ray imaging system according to the first embodiment includes an X-ray generating apparatus 101, an X-ray control apparatus 102, an imaging control apparatus 103, and an X-ray imaging apparatus 104.

The X-ray generating apparatus 101 generates X-rays and exposes them to an object. The X-ray control apparatus 102 controls generation of X-rays in the X-ray generating apparatus 101. The imaging control apparatus 103 includes, for example, one or a plurality of processors (CPUs) and a memory, and the processor executes a program stored in the memory to obtain an X-ray image and perform image processing. Note that each of processes including the image processing performed by the imaging control apparatus 103 may be implemented by dedicated hardware or by cooperation of hardware and software. The X-ray imaging apparatus 104 includes a phosphor 105 that converts X-rays into visible light, and a two-dimensional detector 106 that detects visible light. The two-dimensional detector is a sensor in which pixels 20 for detecting X-ray quanta are arranged in an array of X columns×Y rows, and outputs image information.

The imaging control apparatus 103 functions as an image processing apparatus that processes a radiation image by the above-described processor. An obtaining unit 131, a correcting unit 132, a signal processing unit 133, and an image processing unit 134 indicate examples of the functional components of an image processing apparatus. The obtaining unit 131 obtains a plurality of radiation images corresponding to a plurality of different radiation energies obtained by irradiating an object with radiation and performing imaging. The correcting unit 132 generates a plurality of images to be used for energy subtraction processing by correcting the plurality of radiation images obtained by the obtaining unit 131. Furthermore, the correcting unit 132 performs correction for reducing at least one of an error caused depending on a dose, an error caused depending on the thickness of the object, and an error caused depending on radiation energy. Details of the correcting unit 132 will be described later. The signal processing unit 133 generates a material characteristic image using the plurality of images generated by the correcting unit 132. The material characteristic image is an image obtained in the energy subtraction processing, such as a material decomposition image separately representing materials like a bone and a soft tissue, and a material identification image representing an effective atomic number and its area density. Details of the signal processing unit 133 will be described later. The image processing unit 134 generates a virtual monochromatic X-ray image using the obtained material characteristic image. Details of the image processing unit 134 will be described later.

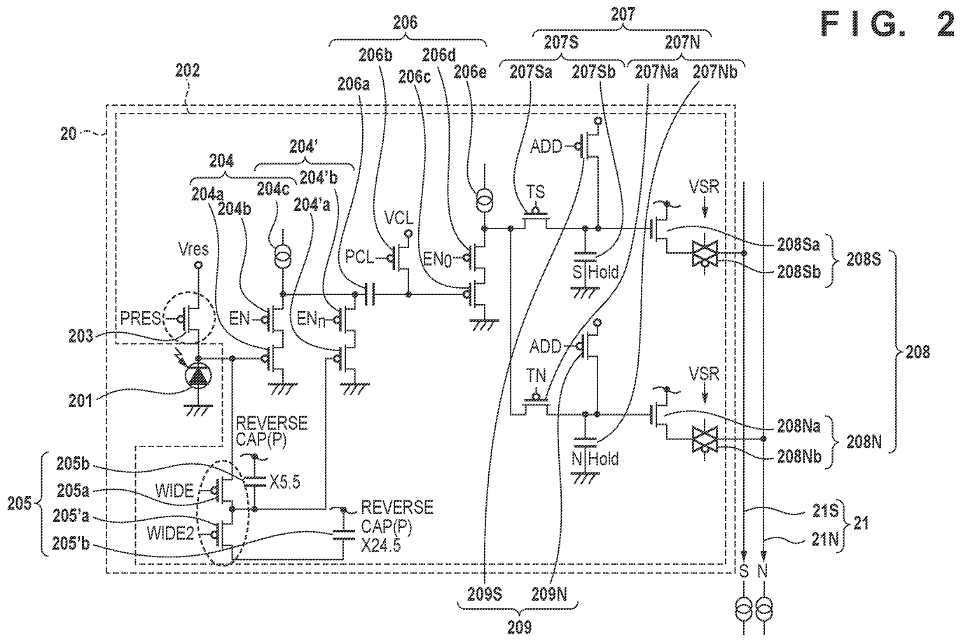

FIG. 2 is an equivalent circuit diagram of the pixel 20 according to the first embodiment. The pixel 20 includes a photoelectric conversion element 201 and an output circuit unit 202. The photoelectric conversion element 201 can typically be a photodiode. The output circuit unit 202 includes an amplification circuit unit 204, a clamp circuit unit 206, a sample and hold circuit unit 207, and a selection circuit unit 208.

The photoelectric conversion element 201 includes a charge accumulation portion. The charge accumulation portion is connected to the gate of a MOS transistor 204a of the amplification circuit unit 204. The source of the MOS transistor 204a is connected to a current source 204c via a MOS transistor 204b. The MOS transistor 204a and the current source 204c form a source follower circuit. The MOS transistor 204b is an enable switch that is turned on when an enable signal EN supplied to its gate is set at an active level, and sets the source follower circuit in an operation state.

In the example shown in FIG. 2, the charge accumulation portion of the photoelectric conversion element 201 and the gate of the MOS transistor 204a form a common node, and this node functions as a charge-voltage converter that converts charges accumulated in the charge accumulation portion into a voltage. That is, a voltage V (=Q/C) determined by charges Q accumulated in the charge accumulation portion and a capacitance value C of the charge-voltage converter appears in the charge-voltage converter. The charge-voltage converter is connected to a reset potential Vres via a reset switch 203. When a reset signal PRES is set at an active level, the reset switch 203 is turned on, and the potential of the charge-voltage converter is reset to the reset potential Vres.

The clamp circuit unit 206 clamps, by a clamp capacitor 206a, noise output from the amplification circuit unit 204 in accordance with the reset potential of the charge-voltage converter. That is, the clamp circuit unit 206 is a circuit configured to cancel the noise from a signal output from the source follower circuit in accordance with charges generated by photoelectric conversion in the photoelectric conversion element 201. The noise includes kTC noise at the time of reset. Clamping is performed by turning on a MOS transistor 206b by setting a clamp signal PCL at an active level, and then turning off the MOS transistor 206b by setting the clamp signal PCL at an inactive level. The output side of the clamp capacitor 206a is connected to the gate of a MOS transistor 206c. The source of the MOS transistor 206c is connected to a current source 206e via a MOS transistor 206d. The MOS transistor 206c and the current source 206e form a source follower circuit. The MOS transistor 206d is an enable switch that is turned on when an enable signal ENO supplied to its gate is set at an active level, and sets the source follower circuit in an operation state.

The signal output from the clamp circuit unit 206 in accordance with charges generated by photoelectric conversion in the photoelectric conversion element 201 is written, as an optical signal, in a capacitor 207Sb via a switch 207Sa when an optical signal sampling signal TS is set at an active level. The signal output from the clamp circuit unit 206 when turning on the MOS transistor 206b immediately after resetting the potential of the charge-voltage converter is a clamp voltage. The noise signal is written in a capacitor 207Nb via a switch 207Na when a noise sampling signal TN is set at an active level. This noise signal includes an offset component of the clamp circuit unit 206. The switch 207Sa and the capacitor 207Sb form a signal sample and hold circuit 207S, and the switch 207Na and the capacitor 207Nb form a noise sample and hold circuit 207N. The sample and hold circuit unit 207 includes the signal sample and hold circuit 207S and the noise sample and hold circuit 207N.

When a driving circuit unit drives a row selection signal to an active level, the signal (optical signal) held in the capacitor 207Sb is output to a signal line 21S via a MOS transistor 208Sa and a row selection switch 208Sb. In addition, the signal (noise) held in the capacitor 207Nb is simultaneously output to a signal line 21N via a MOS transistor 208Na and a row selection switch 208Nb. The MOS transistor 208Sa forms a constant current source and a source follower circuit (not shown) provided in the signal line 21S. Similarly, the MOS transistor 208Na forms a constant current source and a source follower circuit (not shown) provided in the signal line 21N. The MOS transistor 208Sa and the row selection switch 208Sb form a signal selection circuit unit 208S, and the MOS transistor 208Na and the row selection switch 208Nb form a noise selection circuit unit 208N. The selection circuit unit 208 includes the signal selection circuit unit 208S and the noise selection circuit unit 208N.

The pixel 20 may include an addition switch 209S that adds the optical signals of the plurality of adjacent pixels 20. In an addition mode, an addition mode signal ADD is set at an active level, and the addition switch 209S is turned on. This causes the addition switch 209S to interconnect the capacitors 207Sb of the adjacent pixels 20, and the optical signals are averaged. Similarly, the pixel 20 may include an addition switch 209N that adds noise components of the plurality of adjacent pixels 20. When the addition switch 209N is turned on, the capacitors 207Nb of the adjacent pixels 20 are interconnected by the addition switch 209N, thereby averaging the noise components. An adder 209 includes the addition switches 209S and 209N.

Furthermore, the pixel 20 may include a sensitivity changing unit 205 for changing the sensitivity. The pixel 20 can include, for example, a first sensitivity change switch 205a, a second sensitivity change switch 205′a, and their circuit elements. When a first change signal WIDE is set at an active level, the first sensitivity change switch 205a is turned on to add the capacitance value of a first additional capacitor 205b to the capacitance value of the charge-voltage converter. This decreases the sensitivity of the pixel 20. When a second change signal WIDE2 is set at an active level, the second sensitivity change switch 205′a is turned on to add the capacitance value of a second additional capacitor 205′b to the capacitance value of the charge-voltage converter. This further decreases the sensitivity of the pixel 20. In this way, it is possible to receive a larger light amount by adding a function of decreasing the sensitivity of the pixel 20, thereby widening a dynamic range. When the first change signal WIDE is set at the active level, an enable signal ENw may be set at an active level to cause a MOS transistor 204′a to perform a source follower operation instead of the MOS transistor 204a.

The X-ray imaging apparatus 104 reads out the output of the above-described pixel circuit from the two-dimensional detector 106, and causes an A/D converter (not shown) to convert the output into a digital value, thereby transferring an image to the imaging control apparatus 103.

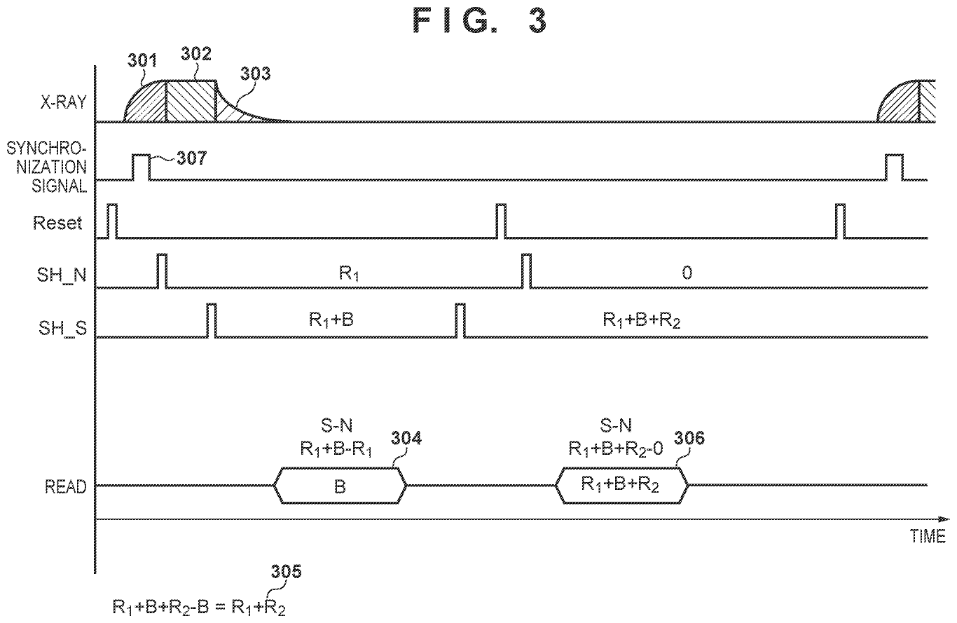

The operation of the X-ray imaging system having the above-described arrangement according to the first embodiment will be described next. FIG. 3 shows the driving timing of the X-ray imaging apparatus 104 when obtaining a plurality of X-ray images of different energies to be provided to energy subtraction in the X-ray imaging system according to the first embodiment. When the abscissa represents the time, waveforms in FIG. 3 indicate timings of X-ray exposure, a synchronization signal, reset of the photoelectric conversion element 201, the sample and hold circuit 207, and readout of an image from a signal line 21.

After the reset signal resets the photoelectric conversion element 201, X-rays are exposed. The tube voltage of the X-rays ideally has a rectangular waveform but it takes a finite time for the tube voltage to rise or fall. Especially, if pulsed X-rays are used and the exposure time is short, the tube voltage is not considered to have a rectangular waveform any more, and has waveforms, as indicated by X-rays 301 to 303. The X-rays 301 during the rising period, the X-rays 302 during the stable period, and the X-rays 303 during the falling period have different X-ray energies. Therefore, by obtaining an X-ray image corresponding to radiation during a period divided by a sample and hold operation, a plurality of kinds of X-ray images of different energies are obtained.

The X-ray imaging apparatus 104 causes the noise sample and hold circuit 207N to perform sampling after exposure of the X-rays 301 during the rising period, and causes the signal sample and hold circuit 207S to perform sampling after exposure of the X-rays 302 during the stable period. After that, the X-ray imaging apparatus 104 reads out, as an image, the difference between the signal lines 21N and 21S. At this time, a signal (R₁) of the X-rays 301 during the rising period is held in the noise sample and hold circuit 207N, and the sum (R₁+B) of the signal of the X-rays 301 during the rising period and a signal (B) of the X-rays 302 during the stable period is held in the signal sample and hold circuit 207S. Therefore, an image 304 corresponding to the signal of the X-rays 302 during the stable period is read out.

Next, after completion of exposure of the X-rays 303 during the falling period and readout of the image 304, the X-ray imaging apparatus 104 causes the signal sample and hold circuit 207S to perform sampling again. After that, the X-ray imaging apparatus 104 resets the photoelectric conversion element 201, causes the noise sample and hold circuit 207N to perform sampling again, and reads out, as an image, the difference between the signal lines 21N and 21S. At this time, a signal in a state in which no X-rays are exposed is held in the noise sample and hold circuit 207N, and the sum (R₁+B+R₂) of the signal of the X-rays 301 during the rising period, the signal of the X-rays 302 during the stable period, and a signal (R₂) of the X-rays 303 during the falling period is held in the signal sample and hold circuit 207S. Therefore, an image 306 corresponding to the signal of the X-rays 301 during the rising period, the signal of the X-rays 302 during the stable period, and the signal of the X-rays 303 during the falling period is read out. After that, by calculating the difference between the images 306 and 304, an image 305 corresponding to the sum of the X-rays 301 during the rising period and the X-rays 303 during the falling period is obtained. This calculation processing may be performed by the X-ray imaging apparatus 104 or the imaging control apparatus 103.

The timing of resetting the sample and hold circuit 207 and the photoelectric conversion element 201 is decided using a synchronization signal 307 indicating the start of exposure of X-rays from the X-ray generating apparatus 101. As a method of detecting the start of exposure of X-rays, an arrangement for measuring the tube current of the X-ray generating apparatus 101 and determining whether the current value exceeds a preset threshold can be used but the present invention is not limited to this. For example, an arrangement for detecting the start of exposure of X-rays by repeatedly reading out the pixel 20 and determining whether the pixel value exceeds a preset threshold after completion of the reset of the photoelectric conversion element 201 may be used. Alternatively, for example, an arrangement for detecting the start of exposure of X-rays by incorporating an X-ray detector different from the two-dimensional detector 106 in the X-ray imaging apparatus 104 and determining whether a measured value of the X-ray detector exceeds a preset threshold may be used. In either method, after a time designated in advance elapses after the input of the synchronization signal 307 indicating the start of exposure of X-rays, sampling of the signal sample and hold circuit 207S, sampling of the noise sample and hold circuit 207N, and reset of the photoelectric conversion element 201 are performed.

As described above, the image 304 corresponding to the stable period of the pulsed X-rays and the image 305 corresponding to the sum of the signal during the rising period and that during the falling period are obtained. Since the energies of the X-rays exposed when forming the two X-ray images are different, calculation is performed for the X-ray images, thereby making it possible to perform energy subtraction processing.

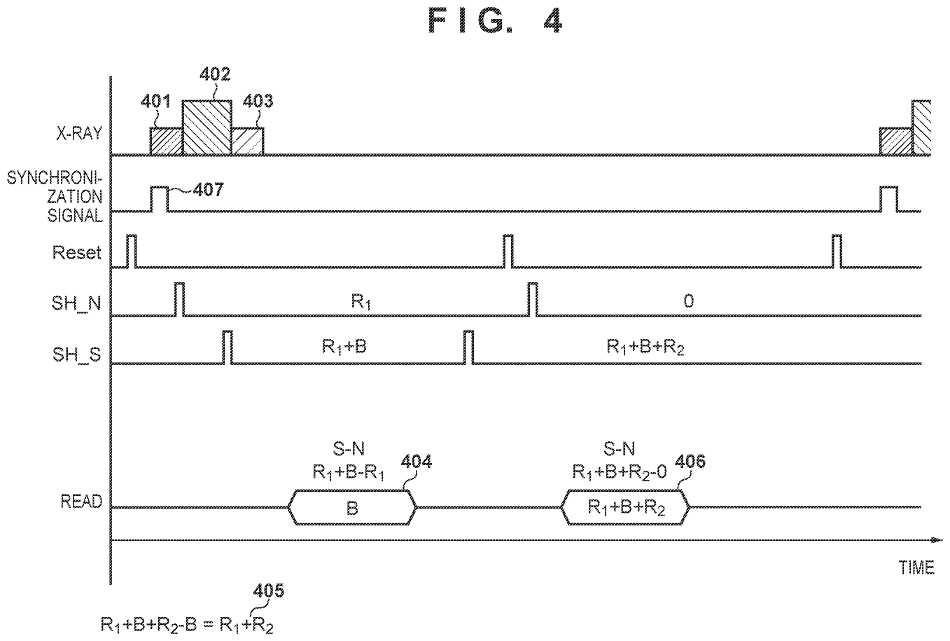

FIG. 4 shows the driving timing, different from FIG. 3, of the X-ray imaging apparatus 104 when obtaining a plurality of X-ray images of different energies to be provided to energy subtraction in the X-ray imaging system according to the first embodiment. FIG. 4 is different from FIG. 3 in that the tube voltage of the X-ray generating apparatus 101 is actively switched.

First, after the reset of the photoelectric conversion element 201, the X-ray generating apparatus 101 exposes low energy X-rays 401. In this state, the X-ray imaging apparatus 104 causes the noise sample and hold circuit 207N to perform sampling. After that, the X-ray generating apparatus 101 switches the tube voltage to expose high energy X-rays 402. In this state, the X-ray imaging apparatus 104 causes the signal sample and hold circuit 207S to perform sampling. After that, the X-ray generating apparatus 101 switches the tube voltage to expose low energy X-rays 403. The X-ray imaging apparatus 104 reads out, as an image, the difference between the signal lines 21N and 21S. At this time, a signal (R₁) of the low energy X-rays 401 is held in the noise sample and hold circuit 207N, and the sum (R₁+B) of the signal of the low energy X-rays 401 and a signal (B) of the high energy X-rays 402 is held in the signal sample and hold circuit 207S. Therefore, an image 404 corresponding to the signal of the high energy X-rays 402 is read out.

Next, after completion of the exposure of the low energy X-rays 403 and the readout of the image 404, the X-ray imaging apparatus 104 causes the signal sample and hold circuit 207S to perform sampling again. After that, the X-ray imaging apparatus 104 resets the photoelectric conversion element 201, causes the noise sample and hold circuit 207N to perform sampling again, and reads out, as an image, the difference between the signal lines 21N and 21S. At this time, a signal in a state in which no X-rays are exposed is held in the noise sample and hold circuit 207N, and the sum (R₁+B+R₂) of the signal of the low energy X-rays 401, the signal of the high energy X-rays 402, and a signal (R₂) of the low energy X-rays 403 is held in the signal sample and hold circuit 207S. Therefore, an image 406 corresponding to the signal of the low energy X-rays 401, the signal of the high energy X-rays 402, and the signal of the low energy X-rays 403 is read out.

After that, by calculating the difference between the images 406 and 404, an image 405 corresponding to the sum of the low energy X-rays 401 and the low energy X-rays 403 is obtained. This calculation processing may be performed by the X-ray imaging apparatus 104 or the imaging control apparatus 103. With respect to a synchronization signal 407, the same as in FIG. 3 applies. As described above, by obtaining images while actively switching the tube voltage, the energy difference between radiation images of low energy and high energy can be made large, as compared with the method shown in FIG. 3.

Next, energy subtraction processing by the imaging control apparatus 103 will be described. The energy subtraction processing according to the first embodiment is divided into three stages of correction processing by the correcting unit 132, signal processing by the signal processing unit 133, and image processing by the image processing unit 134. Each process will be described below.

(Explanation of Correction Processing)

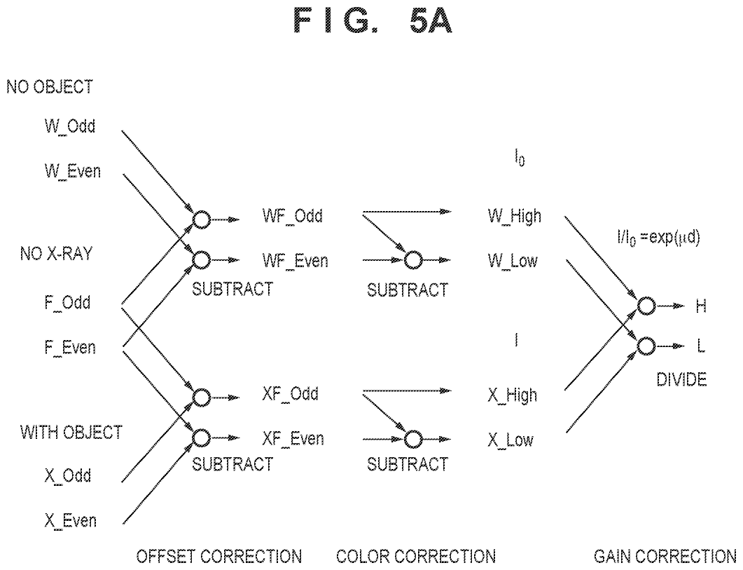

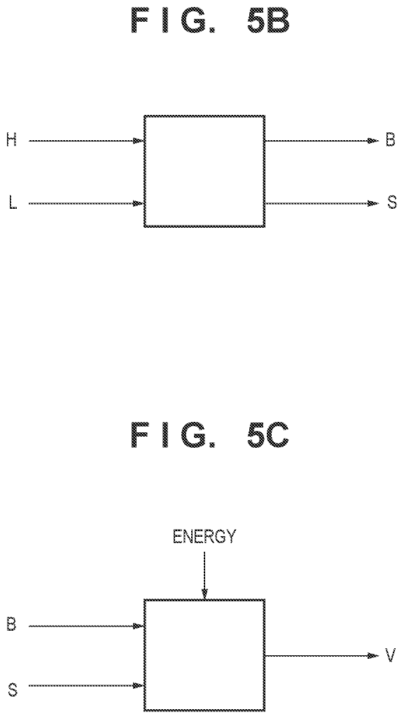

The correction processing is processing of generating, by processing a plurality of radiation images obtained from the X-ray imaging apparatus 104, a plurality of images to be used for the signal processing (to be described later) in the energy subtraction processing. FIG. 5A shows the correction processing for the energy subtraction processing according to the first embodiment. First, the obtaining unit 131 causes the X-ray imaging apparatus 104 to perform imaging in a state in which no X-rays are exposed, thereby obtaining images by the driving operation shown in FIG. 3 or 4. With this driving operation, two images are read out. The first image (image 304 or 404) will be referred to as F_ODD hereinafter and the second image (image 306 or 406) will be referred to as F_EVEN hereinafter. Each of F_ODD and F_EVEN is an image corresponding to FPN (Fixed Pattern Noise) of the X-ray imaging apparatus 104.

Next, the obtaining unit 131 causes the X-ray imaging apparatus 104 to perform imaging by exposing X-rays in a state in which there is no object, thereby obtaining gain correction images output from the X-ray imaging apparatus 104 by the driving operation shown in FIG. 3 or 4. With this driving operation, two images are read out, similar to the above operation. The first gain correction image (image 304 or 404) will be referred to as W_ODD hereinafter and the second gain correction image (image 306 or 406) will be referred to as W_EVEN hereinafter. Each of W_ODD and W_EVEN is an image corresponding to the sum of the FPN of the X-ray imaging apparatus 104 and the signal by X-rays. The correcting unit 132 subtracts F_ODD from W_ODD and F_EVEN from W_EVEN, thereby obtaining images WF_ODD and WF_EVEN from each of which the FPN of the X-ray imaging apparatus 104 has been removed. This is called offset correction.

WF_ODD is an image corresponding to the X-rays 302 during the stable period, and WF_EVEN is an image corresponding to the sum of the X-rays 301 during the rising period, the X-rays 302 during the stable period, and the X-rays 303 during the falling period. Therefore, the correcting unit 132 obtains an image corresponding to the sum of the X-rays 301 during the rising period and the X-rays 303 during the falling period by subtracting WF_ODD from WF_EVEN. The processing of obtaining an image corresponding to X-rays during a specific period divided by the sample and hold operation by subtraction of a plurality of images is called color correction. The energy of the X-rays 301 during the rising period and that of the X-rays 303 during the falling period are lower than the energy of the X-rays 302 during the stable period. Therefore, by subtracting WF_ODD from WF_EVEN by color correction, a low energy image W_Low when there is no object is obtained. Furthermore, a high energy image W_High when there is no object is obtained from WF_ODD.

Next, the obtaining unit 131 causes the X-ray imaging apparatus 104 to perform imaging by exposing X-rays in a state in which there is an object, thereby obtaining images output from the X-ray imaging apparatus 104 by the driving operation shown in FIG. 3 or 4. At this time, two images are read out. The first image (image 304 or 404) will be referred to as X_ODD hereinafter and the second image (image 306 or 406) will be referred to as X_EVEN hereinafter. The correcting unit 132 performs the same offset correction processing and color correction processing as those when there is no object, thereby obtaining a low energy image X_Low when there is the object and a high energy image X_High when there is the object.

When d represents the thickness of the object, μ represents the linear attenuation coefficient of the object, Jo represents the output of the pixel 20 when there is no object, and I represents the output of the pixel 20 when there is the object, equation (1) below holds.

I=I₀ exp(−μd) (1)

Equation (1) is modified to obtain equation (2) below. The right-hand side of equation (2) represents the attenuation rate of the object. The attenuation rate of the object is a real number between 0 and 1.

I/I₀=exp(−μd) (2)

Therefore, the correcting unit 132 obtains an attenuation rate image L at low energy by dividing the low energy image X_Low when there is the object by the low energy image W_Low when there is no object. Similarly, the correcting unit 132 obtains an attenuation rate image H at high energy by dividing the high energy image X_High when there is the object by the high energy image W_High when there is no object. The processing of obtaining an attenuation rate image by dividing an image obtained based on a radiation image obtained when there is an object by an image obtained based on a radiation image obtained when there is no object is called gain correction. The correction processing (except for dose dependence correction to be described later) by the correcting unit 132 according to the first embodiment has been described above.

(Explanation of Signal Processing)

FIG. 5B is a block diagram of the signal processing of the energy subtraction processing according to the first embodiment. The signal processing unit 133 generates a material characteristic image using a plurality of images obtained from the correcting unit 132. Generation of a material characteristic image formed from a bone thickness image B and a soft tissue thickness image S will be described below. The signal processing unit 133 performs the following processing to obtain the bone thickness image B and the soft tissue thickness image S from the attenuation rate image L at low energy and the attenuation rate image H at high energy, both of which have been obtained by the correction processing shown in FIG. 5A.

First, when E represents the energy of X-ray photons, N(E) represents the number of photons at the energy E, B represents a bone thickness, S represents a soft tissue thickness, μ_{B}(E) represents the linear attenuation coefficient of the bone at the energy E, μ_{S}(E) represents the linear attenuation coefficient of the soft tissue at the energy E, and I/I₀ represents the attenuation rate, equation (3) below holds.

$$I / {I}_{0} = \frac{{{\int }^{∞}}_{0} N ( E ) exp \{ - {μ}_{B} ( E ) B - {μ}_{S} ( E ) S \} E d E}{{{\int }^{∞}}_{0} N ( E ) E d E} \qquad ( 3 )$$

The number N(E) of photons at the energy E is an X-ray spectrum. The X-ray spectrum is obtained by simulation or actual measurement. Each of the linear attenuation coefficient μ_{B}(E) of the bone at the energy E and the linear attenuation coefficient μ_{S}(E) of the soft tissue at the energy E is obtained from a database of NIST (National Institute of Standards and Technology) or the like. Therefore, according to equation (3), it is possible to calculate the attenuation rate I/I₀ for the arbitrary bone thickness B, soft tissue thickness S, and X-ray spectrum N(E).

When N_{L}(E) represents a low energy X-ray spectrum and N_{H}(E) represents a high energy X-ray spectrum, equations (4) below hold. Note that L represents a pixel value in the attenuation rate image at low energy and H represents a pixel value in the attenuation rate image at high energy.

$$L = \frac{{{\int }_{0}}^{∞} {N}_{L} ( E ) exp \{ - {μ}_{B} ( E ) B - {μ}_{S} ( E ) S \} E d E}{{{\int }^{∞}}_{0} {N}_{L} ( E ) E d E} \\ H = \frac{{{\int }^{∞}}_{0} {N}_{H} ( E ) exp \{ - {μ}_{B} ( E ) B - {μ}_{S} ( E ) S \} E d E}{{{\int }^{∞}}_{0} {N}_{H} ( E ) E d E} \qquad ( 4 )$$

By solving nonlinear simultaneous equations (4), the bone thickness B and the soft tissue thickness S are obtained. A case in which the Newton-Raphson method is used as a representative method of solving the nonlinear simultaneous equations will be explained. When m represents an iteration count of the Newton-Raphson method, B^{m} represents a bone thickness after the mth iteration, and S^{m} represents a soft tissue thickness after the mth iteration, an attenuation rate H^{m} at high energy after the mth iteration and an attenuation rate L^{m} at low energy after the mth iteration are given by:

$${L}^{m} = \frac{{{\int }^{∞}}_{0} {N}_{L} ( E ) exp \{ - {μ}_{B} ( E ) {B}^{m} - {μ}_{S} ( E ) {S}^{m} \} E d E}{{{\int }^{∞}}_{0} {N}_{L} ( E ) E d E} \\ {H}^{m} = \frac{{{\int }^{∞}}_{0} {N}_{H} ( E ) exp \{ - {μ}_{B} ( E ) {B}^{m} - {μ}_{S} ( E ) {S}^{m} \} E d E}{{{\int }^{∞}}_{0} {N}_{H} ( E ) E d E} \qquad ( 5 )$$

The change rates of the attenuation rates when the thicknesses slightly change are given by:

$$\frac{\partial {H}^{m}}{\partial {B}^{m}} = \frac{{{\int }^{∞}}_{0} - {μ}_{B} ( E ) {N}_{H} ( E ) exp \{ - {μ}_{B} ( E ) {B}^{m} - {μ}_{S} ( E ) {S}^{m} \} E d E}{{{\int }^{∞}}_{0} {N}_{H} ( E ) E d E} \\ \frac{\partial {L}^{m}}{\partial {B}^{m}} = \frac{{{\int }^{∞}}_{0} - {μ}_{B} ( E ) {N}_{L} ( E ) exp \{ - {μ}_{B} ( E ) {B}^{m} - {μ}_{S} ( E ) {S}^{m} \} E d E}{{{\int }^{∞}}_{0} {N}_{L} ( E ) E d E} \\ \frac{\partial {H}^{m}}{\partial {S}^{m}} = \frac{{{\int }^{∞}}_{0} - {μ}_{S} ( E ) {N}_{H} ( E ) exp \{ - {μ}_{B} ( E ) {B}^{m} - {μ}_{S} ( E ) {S}^{m} \} E d E}{{{\int }^{∞}}_{0} {N}_{H} ( E ) E d E} \\ \frac{\partial {L}^{m}}{\partial {S}^{m}} = \frac{{{\int }^{∞}}_{0} - {μ}_{S} ( E ) {N}_{L} ( E ) exp \{ - {μ}_{B} ( E ) {B}^{m} - {μ}_{S} ( E ) {S}^{m} \} E d E}{{{\int }^{∞}}_{0} {N}_{L} ( E ) E d E} \qquad ( 6 )$$

At this time, using the attenuation rate H at high energy and the attenuation rate L at low energy, a bone thickness B^{m+1} and a soft tissue thickness S^{m+1} after the (m+1)th iteration are given by:

$$[ \begin{matrix}{B}^{m + 1} \\ {S}^{m + 1}\end{matrix} ] = [ \begin{matrix}{B}^{m} \\ {S}^{m}\end{matrix} ] + {[ \begin{matrix}\frac{\partial {H}^{m}}{\partial {B}^{m}} & \frac{\partial {H}^{m}}{\partial {S}^{m}} \\ \frac{\partial {L}^{m}}{\partial {B}^{m}} & \frac{\partial {L}^{m}}{\partial {S}^{m}}\end{matrix} ]}^{- 1} [ \begin{matrix}H - {H}^{m} \\ L - {L}^{m}\end{matrix} ] \qquad ( 7 )$$

When det represents a determinant, the inverse matrix of a 2×2 matrix is given, using the Cramer's rule, by:

$$det = \frac{\partial {H}^{m}}{\partial {B}^{m}} \frac{\partial {L}^{m}}{\partial {S}^{m}} - \frac{\partial {H}^{m}}{\partial {S}^{m}} {\frac{\partial {L}^{m}}{\partial {B}^{m}} \\ [ \begin{matrix}\frac{\partial {H}^{m}}{\partial {B}^{m}} & \frac{\partial {H}^{m}}{\partial {S}^{m}} \\ \frac{\partial {L}^{m}}{\partial {B}^{m}} & \frac{\partial {L}^{m}}{\partial {S}^{m}}\end{matrix} ]}^{- 1} = \frac{1}{det} [ \begin{matrix}\frac{\partial {L}^{m}}{\partial {S}^{m}} & - \frac{\partial {H}^{m}}{\partial {S}^{m}} \\ - \frac{\partial {L}^{m}}{\partial {B}^{m}} & \frac{\partial {H}^{m}}{\partial {B}^{m}}\end{matrix} ] \qquad ( 8 )$$

Therefore, by substituting equation (8) into equation (7), equations (9) below are obtained.

$${B}^{m + 1} = {B}^{m} + \frac{1}{det} \frac{\partial {L}^{m}}{\partial {S}^{m}} ( H - {H}^{m} ) - \frac{1}{det} \frac{\partial {H}^{m}}{\partial {S}^{m}} ( L - {L}^{m} ) \\ {S}^{m + 1} = {S}^{m} - \frac{1}{det} \frac{\partial {L}^{m}}{\partial {B}^{m}} ( H - {H}^{m} ) + \frac{1}{det} \frac{\partial {H}^{m}}{\partial {B}^{m}} ( L - {L}^{m} ) \qquad ( 9 )$$

When the above calculation processing is repeated, the difference between the attenuation rate H^{m} at high energy after the mth iteration and the actually measured attenuation rate H at high energy approaches almost 0. The same applies to the attenuation rate L at low energy. This causes the bone thickness B^{m} after the mth iteration to converge to the bone thickness B, and causes the soft tissue thickness S^{m} after the mth iteration to converge to the soft tissue thickness S. As described above, the nonlinear simultaneous equations (4) can be solved. Therefore, by calculating equations (4) for all the pixels, the bone thickness image B and the soft tissue thickness image S can be obtained from the attenuation rate image L at low energy and the attenuation rate image H at high energy.

Note that the bone thickness B and the soft tissue thickness S are calculated in the first embodiment but the present invention is not limited to this. For example, a water thickness W and a contrast agent thickness I may be calculated. That is, decomposition may be performed into the thicknesses of arbitrary two kinds of materials. An image of an effective atomic number Z and an image of an area density D may be obtained from the attenuation rate image L at low energy and the attenuation rate image H at high energy, both of which have been obtained by the correction processing shown in FIG. 5A. The effective atomic number Z indicates the equivalent atomic number of a mixture. The area density D is the product of the density [g/cm³] of the object and the thickness [cm] of the object.

In the first embodiment, the nonlinear simultaneous equations are solved using the Newton-Raphson method. However, the present invention is not limited to this. For example, an iterative method such as a least square method or a bisection method may be used. Furthermore, in the first embodiment, the nonlinear simultaneous equations are solved using the iterative method but the present invention is not limited to this. An arrangement for generating a table by obtaining, in advance, the bone thicknesses B and the soft tissue thicknesses S for various combinations of the attenuation rates H at high energy and the attenuation rates L at low energy, and obtaining the bone thickness B and the soft tissue thickness S at high speed by referring to this table may be used.

(Explanation of Image Processing)

FIG. 5C is a block diagram of the image processing of the energy subtraction processing according to the first embodiment. The image processing unit 134 according to the first embodiment performs image processing of obtaining a virtual monochromatic X-ray image from the bone thickness image B and the soft tissue thickness image S, both of which have been obtained by the signal processing shown in FIG. 5B. The virtual monochromatic X-ray image indicates an image assumed to be obtained when X-rays of single energy are emitted. For example, when E_{V} represents virtual monochromatic X-ray energy, a virtual monochromatic X-ray image V is obtained by:

V=exp{−μ_{B}(E_{V})B−μ_{S}(E_{V})S} (10)

The virtual monochromatic X-ray image is used by Dual Energy CT obtained by combining energy subtraction and three-dimensional reconstruction. At this time, to improve the CNR (Contrast-to-Noise Ratio) of the virtual monochromatic X-ray image, the virtual monochromatic X-ray energy E_{V} is changed. For example, the linear attenuation coefficient μ_{B}(E) of the bone is larger than the linear attenuation coefficient μ_{S}(E) of the soft tissue. However, as the virtual monochromatic X-ray energy E_{V} becomes larger, the difference between μ_{B}(E) and μ_{S}(E) becomes smaller. Therefore, an increase in noise of the virtual monochromatic X-ray image, caused by noise of the bone image, is suppressed. On the other hand, as the virtual monochromatic X-ray energy E_{V} becomes smaller, the difference between μ_{B}(E) and μ_{S}(E) becomes larger, and thus the contrast of the virtual monochromatic X-ray image becomes large. That is, there exists an appropriate value for the energy E_{V} of the virtual monochromatic X-ray image.

Note that the virtual monochromatic X-ray image is generated from the bone thickness B and the soft tissue thickness S in the first embodiment but the present invention is not limited to this. As described above, after calculating the effective atomic number Z and the area density D, the virtual monochromatic X-ray image may be generated using the effective atomic number Z and the area density D. Alternatively, a composite X-ray image may be generated by compositing a plurality of virtual monochromatic X-ray images generated by a plurality of energies E_{V}. The composite X-ray image indicates an image assumed to be obtained when X-rays of an arbitrary spectrum is emitted.

Although the virtual monochromatic X-ray image is generated in the image processing according to the first embodiment, the present invention is not limited to this. The bone thickness image B and the soft tissue thickness image S may be displayed intact. Alternatively, an image obtained by applying a filter in a time direction such as a recursive filter or a filter in a spatial direction such as a Gaussian filter to the bone thickness image B and the soft tissue thickness image S may be displayed. A DSA (Digital Subtraction Angiography) image of the bone may be obtained using images (attenuation rates) at low energy and images (attenuation rates) at high energy before and after contrast agent is injected, and displayed. That is, the image processing according to this embodiment can be said as processing of performing arbitrary calculation for the image after the signal processing.

Note that the DSA image is obtained, for example, as follows. Before injecting the contrast agent, an attenuation rate image L_{M} at low energy and an attenuation rate image H_{M} at high energy are obtained by performing X-ray imaging. Then, a mask image B_{M} of the bone thickness and a mask image S_{M} of the soft tissue thickness are obtained from the images L_{M} and H_{M}. Next, a live image BL of the bone thickness and a live image S_{L} of the soft tissue thickness are obtained from an attenuation rate live image L_{L} at low energy and an attenuation rate live image H_{L} at high energy, both of which have been captured after injecting the contrast agent. A DSA image B_{OSA} of the bone is obtained by subtracting the mask image B_{M} of the bone thickness from the live image BL of the bone thickness.

The correction processing, the signal processing, and the image processing according to this embodiment have been described above. Next, processing of reducing an error occurring in the estimated value, which is executed by the correcting unit 132, will be described. As described with reference to FIGS. 5A to 5C, the energy subtraction processing according to the first embodiment is formed by three steps of the correction processing, the signal processing, and the image processing. At this time, each of the bone thickness B and the soft tissue thickness S obtained by solving equations (4) is set as the estimated value of the thickness. A thickness measured by a ruler or the like is set as the true value of the thickness. If the correction processing and the signal processing are performed appropriately, the estimated value of the thickness should match the true value of the thickness. However, as a result of examination by the present inventors, it is found that the estimated value of the thickness obtained by the above energy subtraction processing does not always match the true value of the thickness. If the error between the estimated value of the thickness and its true value is large, for example, an artifact occurs in the image after the image processing.

As a result of examination by the present inventors, it is found that factors of the error are scattered rays, the dose dependence of the attenuation rate, the thickness dependence of the attenuation rate, the spectrum dependence of the attenuation rate, and the like. In this embodiment, for the sake of descriptive simplicity, it is assumed that the scattered rays have been removed or corrected, and a method of reducing the error caused by a factor other than the scattered rays will be described. That is, the correcting unit 132 includes processing of correcting the attenuation rate or the pixel value of each pixel of an image obtained in the process (FIG. 5A) of generating a plurality of images so as to reduce at least one of an error caused depending on the dose, an error caused depending on the thickness of an object, and an error caused depending on radiation energy. Note that the first embodiment will explain correction associated with the dose dependence of the attenuation rate. Correction associated with the thickness dependence and spectrum dependence of the attenuation rate will be described in the second embodiment.

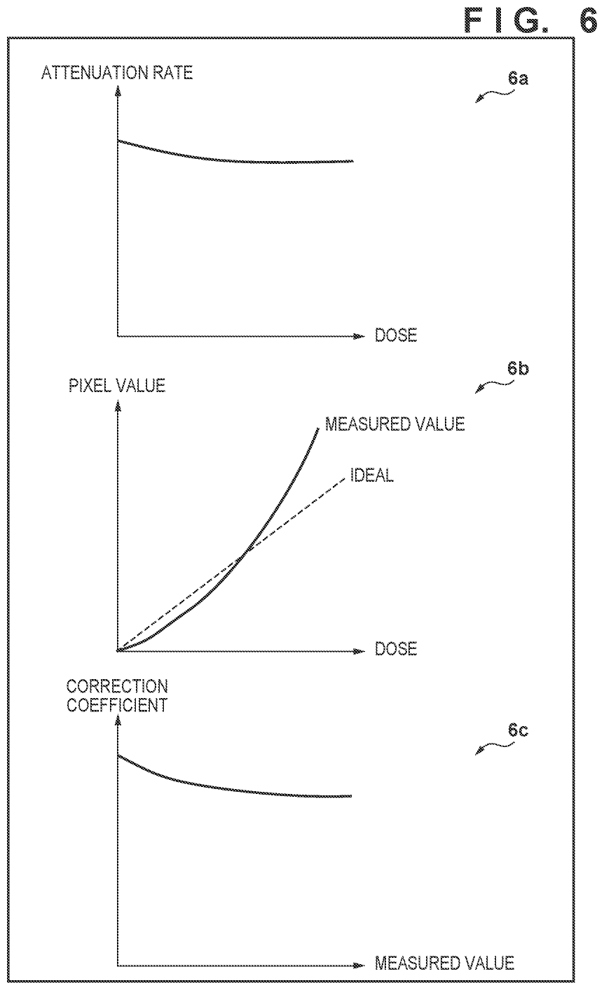

In FIG. 6, a graph 6a shows the dose dependence of the attenuation rate according to the first embodiment. The attenuation rate I/I₀ is given by equation (3). In this example, the spectrum N(E) is multiplied by α (0<α) by adjusting the tube current or exposure time while keeping the tube voltage of the X-ray generating apparatus 101 constant or by adjusting the distance between the X-ray generating apparatus 101 and the X-ray imaging apparatus 104. In this case, both the denominator and numerator of the right-hand side of equation (3) are multiplied by a and the multiplication is canceled. That is, the attenuation rate I/I₀ is considered to be constant even if the dose changes. However, as a result of measuring the attenuation rate while changing only the dose without changing the tube voltage, the filter, or the like, that is, without changing the spectrum, it is found that the attenuation rate is not constant with respect to the change of the dose, as shown in the graph 6a.

In FIG. 6, a graph 6b shows the dose dependence of the pixel value obtained from the X-ray imaging apparatus 104 according to the first embodiment. The pixel value read out from the pixel 20 of the X-ray imaging apparatus 104 should ideally be a value proportional to the dose. However, as a result of measuring the pixel value while changing the dose without changing the tube voltage, the filter, or the like, that is, without changing the spectrum, it is found that the measured value of the pixel value deviates from the ideal straight line, as shown in the graph 6b. The INL (Integral Non-Linearity) of the X-ray imaging apparatus 104 is a factor of preventing the attenuation rate from being constant, as shown in the graph 6a.

In the first embodiment, the attenuation rate is made constant with respect to the change of the dose when the correcting unit 132 performs correction so that the measured value in the graph 6b becomes close to the ideal straight line (predetermined straight line). This correction processing will be referred to as dose dependence correction hereinafter. In FIG. 6, a graph 6c shows a correction coefficient of dose dependence correction according to the first embodiment. In the first embodiment, by multiplying the measured value of the pixel by the correction coefficient shown in the graph 6c, the dose dependence correction makes the measured value in the graph 6b close to the ideal straight line, and the attenuation rate in the graph 6a is almost constant. For example, when x (pixel value x) represents the measured value of the pixel, if g(x) represents the correction coefficient of the pixel value x, a pixel value f(x) after the correction is given by:

f(x)=x*g(x) (11)

It is considered that, for example, the quadratic function of the measured value x of the pixel value is used to approximate the correction coefficient g(x) of the pixel value. That is, when z represents a reference pixel value, the correction coefficient g(x) of the pixel value is given by equation (12) below. With equation (12), the correction coefficient g(z) for the reference pixel value z is 1. Note that coefficients a and b obtained when the quadratic function is used to perform approximation can be obtained from the relationship between the dose and the pixel value in the graph 6b.

g(x)={a(x−z)²+b(x−z)+z}/z (12)

Note that the first embodiment has explained the example of approximating the correction coefficient g(x) of the pixel value by the quadratic function but the present invention is not limited to this. For example, the correction coefficient g(x) of the pixel value may be approximated using an exponential function, logarithm, or the like, or the pixel value f(x) after the correction may be approximated by a cubic function. That is, the pixel value f(x) after the correction or the correction coefficient g(x) of the pixel value may be approximated by an arbitrary function. Alternatively, the relationship between the dose and the measured value may be measured at a plurality of points, and these may be interpolated.

With respect to the function of approximating the pixel value f(x) after the correction or the correction coefficient g(x), the function may be changed for each pixel of the two-dimensional detector 106 or a common function may be used for each of a plurality of regions obtained by dividing the two-dimensional detector 106. For the sake of simplicity of calculation, a common function is preferably used for all the pixels of the two-dimensional detector 106. An arrangement may be adopted to perform correction using a table that returns the pixel value f(x) after the correction when the measured value x of the pixel value is input.

Note that as a method of measuring the relationship between the dose and the pixel value shown in the graph 6b, an arrangement for obtaining the pixel value by performing imaging while changing the exposure time or the tube current of the X-ray generating apparatus 101 can be used. Since the dose is proportional to the product of the tube current and the exposure time, the ideal straight line in the graph 6b is a straight line connecting the origin and the measured value when performing imaging with a given tube current and exposure time. As a method of changing the dose, an arrangement for performing imaging by changing the imaging interval (exposure time) of the two-dimensional detector 106 while the X-ray generating apparatus 101 exposes continuous X-rays by keeping the tube current constant is also preferably used. In this case, the ideal straight line is calculated by assuming that the dose is proportional to the product of the tube current and the imaging interval.

The method of obtaining the correction coefficient g(x) is not limited to the method of measuring the relationship between the dose and the pixel value shown in the graph 6b. For example, as shown in the graph 6a, the relationship between the attenuation rate and the dose is measured when the object is placed and such the correction coefficient g(x) that the attenuation rate is constant regardless of the dose may be defined.

The execution timing of the dose dependence correction in the energy subtraction processing will be described next. Especially, a case in which the above-described dose dependence correction of this embodiment is applied to an arrangement for performing imaging using a sample and hold operation, as shown in FIG. 3 or 4, will be described below. That is, the execution timing of the dose dependence correction in an arrangement for obtaining a plurality of radiation images by performing a sample and hold operation a plurality of times during exposure of radiation of one shot and generating a plurality of images to be provided to the energy subtraction processing will be described.

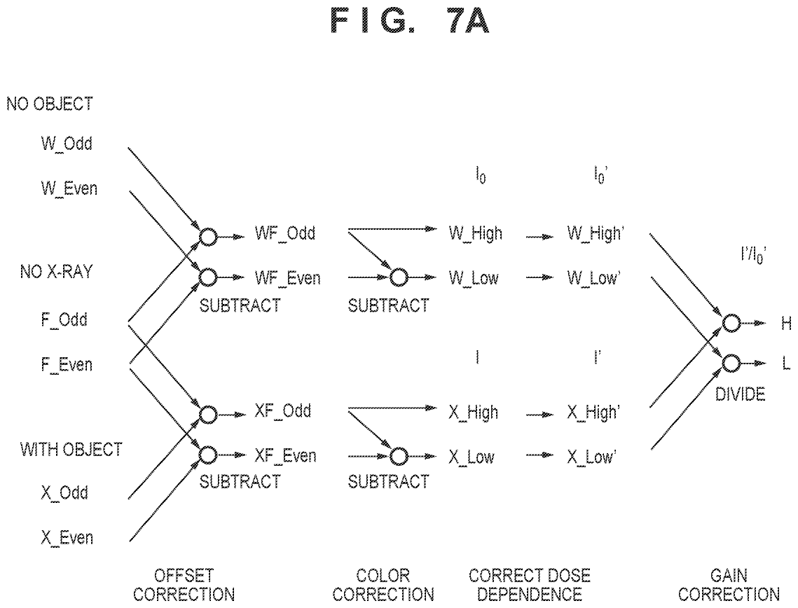

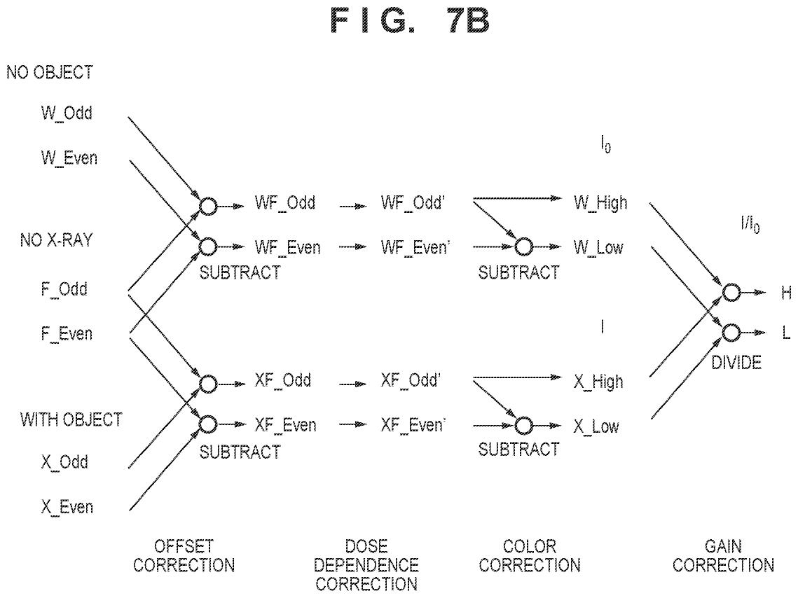

FIG. 7A shows an example of the operation of the correction processing according to the first embodiment. Similar to the correction processing in the energy subtraction processing shown in FIG. 5A, the offset correction and the color correction are performed to obtain the low energy image W_Low when there is no object and the high energy image W_High when there is no object. Similarly, the low energy image X_Low when there is the object and the high energy image X_High when there is the object are obtained. The imaging control apparatus 103 performs the dose dependence correction shown in the graph 6c for these images obtained by performing the offset correction and color correction. In this way, a low energy image W_Low′ when there is no object, a high energy image W_High′ when there is no object, a low energy image X_Low′ when there is the object, and a high energy image X_High′ when there is the object, all of which have undergone the dose dependence correction, are obtained.

After that, the gain correction described with reference to FIG. 5A is performed using the images after the dose dependence correction, thereby obtaining the attenuation rate image L at low energy and the attenuation rate image H at high energy. By performing the dose dependence correction for the images having undergone the color correction, the attenuation rate L at low energy and the attenuation rate H at high energy do not depend on the dose (or dependence is reduced). Therefore, it is expected that the estimated values of the bone thickness B and the soft tissue thickness S calculated by the signal processing shown in FIG. 5B become close to the true values.

Note that in FIG. 7A, the dose dependence correction is performed after execution of the color correction (processes are performed in the order of offset correction→color correction→dose dependence correction→gain correction) but the present invention is not limited to this. For example, as shown in FIG. 7B, the dose dependence correction may be performed before execution of the color correction (processes may be performed in the order of offset correction→dose dependence correction→color correction→gain correction).

Referring to FIG. 7B, WF_ODD represents the image corresponding to the high energy X-rays 402, and WF_EVEN represents the image corresponding to the sum of the low energy X-rays 401, the high energy X-rays 402, and the low energy X-rays 403. Therefore, to obtain the image 405 corresponding to the low energy X-rays, it is necessary to subtract WF_ODD from WF_EVEN, that is, perform the color correction. In FIG. 7B, the dose dependence correction is performed before the color correction. Since WF_EVEN and WF_ODD depend on the dose, it is also preferable to perform the dose dependence correction and then perform the color correction, as shown in FIG. 7B. At this time, the function and coefficient of the dose dependence correction may be different for each of a set of WF_EVEN and WF_ODD and a set of XF_EVEN and XF_ODD. For the sake of simplicity of calculation, an arrangement for performing the dose dependence correction using a function and coefficient common to WF_EVEN, WF_ODD, XF_EVEN, and XF_ODD is preferable.

### Second Embodiment

The first embodiment has explained the dose dependence correction of correcting the dose dependence of an attenuation rate. The second embodiment will describe correction for an error of an attenuation rate depending on a thickness and an error of the attenuation rate depending on a spectrum. Note that the arrangements of an X-ray imaging system and apparatuses forming the system are the same as in the first embodiment.

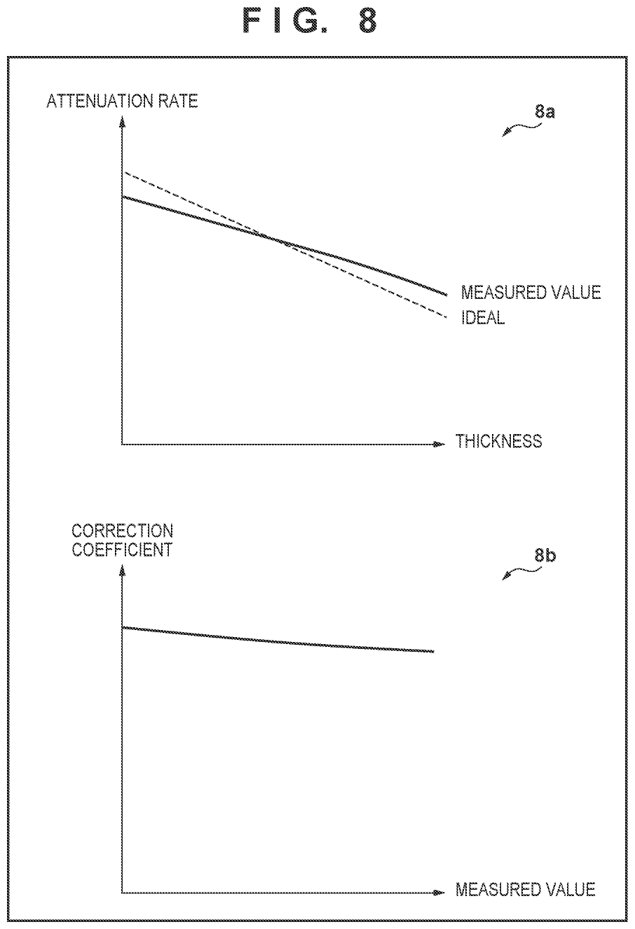

In FIG. 8, a graph 8a shows an example of the thickness dependence of the attenuation rate. By performing the dose dependence correction described in the first embodiment (FIG. 6), the attenuation rate is kept constant even if the dose changes. However, as a result of measuring the attenuation rate while changing the thickness of an object without changing a tube voltage, a filter, or the like, that is, without changing a spectrum, it is found that the measured value of the attenuation rate deviates from an ideal curve or straight line, as shown in the graph 8a. As a factor of the error, a spectrum N(E) used to calculate the attenuation rate indicated by equation (3), a linear attenuation coefficient μ_{B}(E) at energy E, or a linear attenuation coefficient μ_{S}(E) of a soft tissue at the energy E deviates from an actual value.

To prevent this error, a value actually measured using a spectrometer or the like is desirably used as the spectrum N(E). However, X-rays may be absorbed by the exterior of an X-ray imaging apparatus 104 to change the spectrum N(E) or some X-rays pass through a phosphor 105 to change the spectrum N(E). Therefore, to prevent the error, the spectrum N(E) considering X-rays absorbed by or passing through the X-ray imaging apparatus 104 is desirably used. As a result of examination by the present inventor, it is found that even if the above-described spectrum N(E) is modified, an unacceptable error may still exist.

To cope with this, a correcting unit 132 according to the second embodiment corrects the thickness dependence by correcting the attenuation rate of each pixel in an attenuation rate image so that the change of the attenuation rate with respect to the thickness of the object matches the above-described predetermined curve or straight line. In FIG. 8, for example, a graph 8b shows the correction coefficient of the thickness dependence according to the second embodiment. In the second embodiment, correction is performed so that the measured value of the attenuation rate in the graph 8a becomes close to the ideal curve or straight line by multiplying the measured value of the attenuation rate by the correction coefficient shown in the graph 8b. When, for example, y represents the measured value of the attenuation rate, if i(y) represents the correction coefficient of the attenuation rate, a corrected attenuation rate h(y) is given by:

h(y)=y*i(y) (13)

An arrangement of approximating the correction coefficient i(y) of the attenuation rate by the quadratic function of the measured value y of the attenuation rate is preferably used but the present invention is not limited to this. For example, similar to the dose dependence correction, the corrected attenuation rate h(y) or the correction coefficient i(y) of the attenuation rate may be approximated by an arbitrary function. Alternatively, the function may be changed for each pixel of a two-dimensional detector 106 or a common function may be used for each of a plurality of regions obtained by dividing the two-dimensional detector 106. For the sake of simplicity of calculation, an arrangement for using a common function for all the pixels of the two-dimensional detector 106 is preferable.

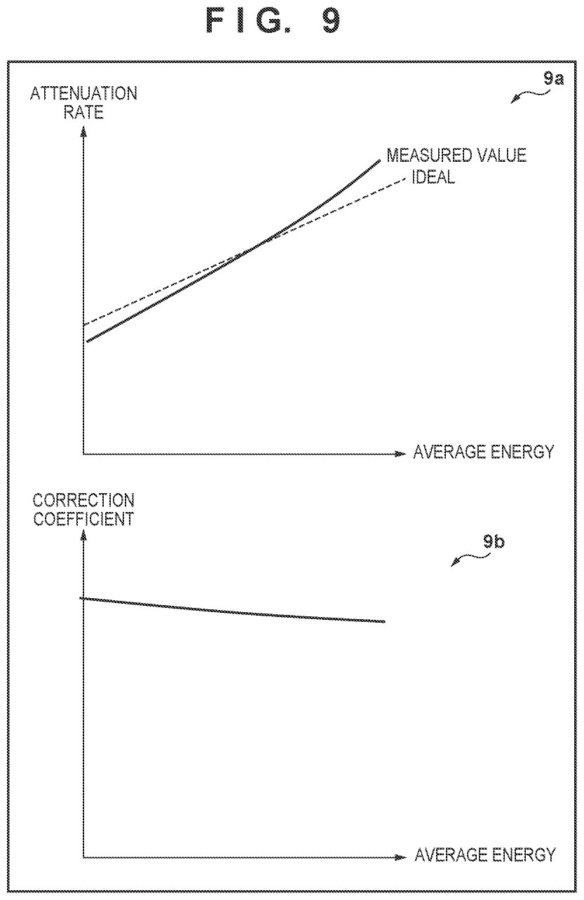

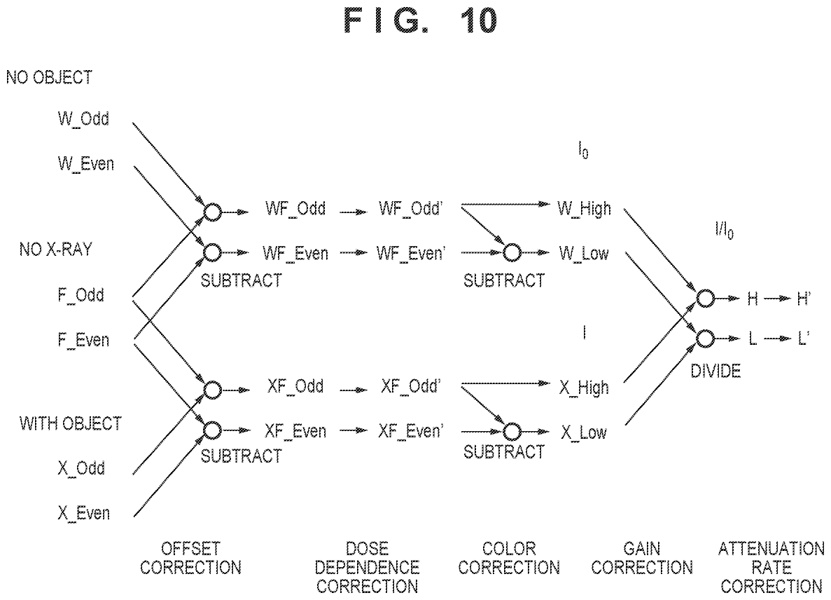

Next, correction for the spectrum dependence of the attenuation rate will be described. In FIG. 9, a graph 9a shows the spectrum dependence of the attenuation rate. By performing the dose dependence correction described in the first embodiment (FIG. 6), the attenuation rate is kept constant even if the dose changes. However, as a result of measuring the attenuation rate while changing the average energy of the spectrum N(E) by changing the tube voltage of X-rays without changing the thickness of the object, it is found that the measured value of the attenuation rate deviates from an ideal curve or straight line, as shown in the graph 9a. A factor of the error is as described above with reference to the graph 8a.

To cope with this, the correcting unit 132 according to the second embodiment corrects the energy dependence to correct the attenuation rate of each pixel in an attenuation rate image so that the change of the attenuation rate with respect to the average energy of radiation matches the predetermined curve or straight line. In FIG. 9, a graph 9b shows the correction coefficient of the spectrum dependence according to the second embodiment. In the second embodiment, correction is performed so that the measured value of the attenuation rate in the graph 9a becomes close to the ideal curve or straight line by multiplying the measured value of the attenuation rate by the correction coefficient shown in the graph 9b. When, for example, y represents the measured value of the attenuation rate and e represents the average energy of the spectrum N(E), if k(e) represents the correction coefficient of the attenuation rate, a corrected attenuation rate j(e) is given by:

j(e)=y*k(e) (14)

Similar to the dose dependence correction, the correction coefficient k(e) of the attenuation rate may be approximated by an arbitrary function. Alternatively, the function may be changed for each pixel of the two-dimensional detector 106 or a common function may be used for each region of the two-dimensional detector 106. For the sake of simplicity of calculation, an arrangement for using a common function for all the pixels of the two-dimensional detector 106 is preferable.

For the sake of descriptive simplicity, the second embodiment has explained the method of individually correcting the thickness dependence and the spectrum dependence. The present invention, however, is not limited to this. For example, the attenuation rate may actually be measured for each combination of a bone thickness B, a soft tissue thickness S, and the spectrum N(E), and such a correction coefficient I(B, S, N(E)) that the attenuation rate matches the ideal curve of the attenuation rate may be obtained, and applied to the measured value of the attenuation rate, thereby performing correction. The ideal curve of attenuation rate is, for example, a curve of an attenuation rate calculated, with respect to an object (in the above description, a bone and a soft tissue) separated or identified in a material characteristic image, from the attenuation coefficient of the object, the thickness, and the X-ray spectrum corresponding to X-ray energy. The attenuation coefficient and the X-ray spectrum can be obtained from a known database. Note that at this time, if the correction coefficient I(B, S, N(E)) is obtained for each of all combinations of the bone thicknesses B, the soft tissue thicknesses S, and the spectra N(E), a data amount becomes enormous. Therefore, a correction coefficient may be obtained, by interpolation, from correction coefficients obtained under a plurality of conditions. The above-described processing will be referred to as attenuation rate correction hereinafter.

The method of performing correction so that the measured value of the attenuation rate in the graph 8a becomes close to the ideal curve or straight line by multiplying the measured value of the attenuation rate by the correction coefficient shown in the graph 8b has been explained above. However, the present invention is not limited to this. For example, the spectrum N(E) may be modified so that the calculated value and the measured value of the attenuation rate match each other. That is, as for a given spectrum N(E), with respect to a plurality of kinds of materials, a plurality of objects having different thicknesses may be captured for each material, and a radiation spectrum may be modified so that a measured attenuation rate matches an attenuation rate calculated by equation (2) or (3). Alternatively, an object of a predetermined material and a predetermined thickness may be captured by a plurality of X-ray energies, and a radiation spectrum may be modified so that the measured attenuation rate matches the attenuation rate calculated by equation (2) or (3). In either case, the modified radiation spectrum is held in an imaging control apparatus 103, and used for signal processing. Therefore, the above-described processing of performing correction for the image having undergone gain correction is unnecessary, the signal processing shown in FIG. 5B need only be executed after performing the correction processing (FIG. 7A or 7B) described in the first embodiment, and the modified spectrum N(E) is used in the signal processing. Note that in measurement of the attenuation rate for modifying the radiation spectrum, it is preferable to obtain an attenuation rate image by performing the correction processing (FIG. 7A or 7B) of correcting the error depending on the dose, which has been described in the first embodiment.

FIG. 10 shows an example of the operation of correction processing performed by the imaging control apparatus 103 according to the second embodiment. As shown in FIG. 7B, correction processes are performed in the order of offset correction→dose dependence correction→color correction→gain correction. Note that the order (offset correction→color correction→dose dependence correction→gain correction) shown in FIG. 7A may be used. In FIG. 10, in addition, attenuation rate correction (correction of the thickness dependence of the attenuation rate and the spectrum dependence of the attenuation rate) is performed after the gain correction, and attenuation rate images H′ and L′ are obtained from attenuation rate images H and L, respectively. By performing the attenuation rate correction, the attenuation rate L at low energy and the attenuation rate H at high energy do not depend on the thickness or spectrum (or the dependence is reduced). Thus, it is expected that the estimated values of the bone thickness B or the soft tissue thickness S calculated by the signal processing shown in FIG. 5B become close to true values.

### Third Embodiment

Each of the first and second embodiments has explained the method of correcting the sensor output and the spectrum. Each of the third and fourth embodiments proposes a calibration method of reducing, by absorption of radiation (X-rays) by a structure other than the object between an X-ray generating apparatus 101 and an X-ray imaging apparatus 104, an error caused by the individual variation of the structure. The third embodiment will exemplify, as an example of the structure influencing absorption of X-rays, a phosphor 105 for converting X-rays into visible light. The arrangement of an imaging system and the equivalent circuit diagram of a pixel 20 according to the third embodiment are the same as in the first embodiment (FIGS. 1 and 2).

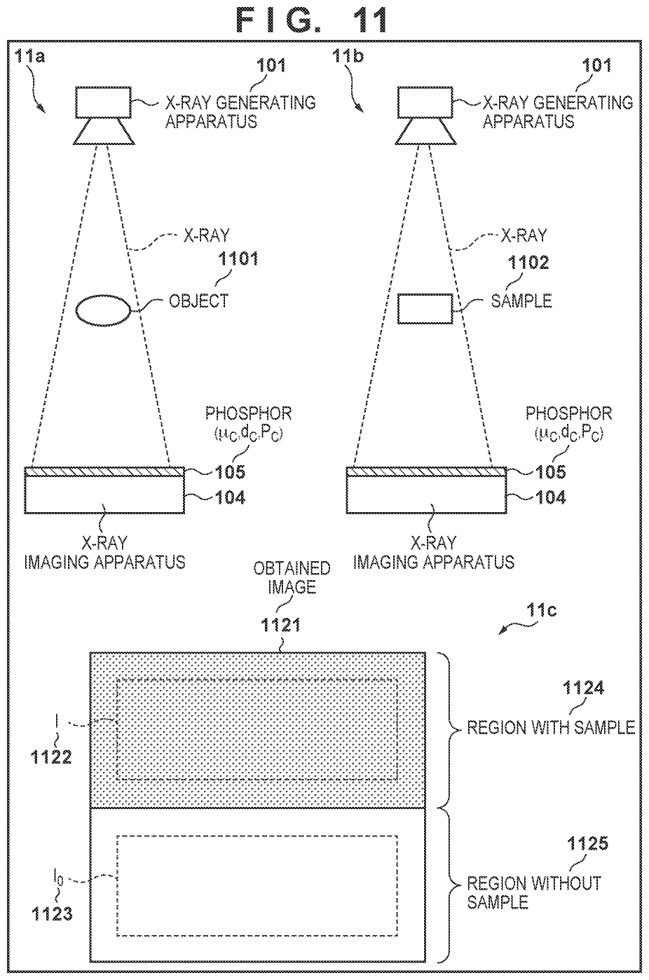

In FIG. 11, an arrangement 11a is an arrangement in X-ray imaging according to the third embodiment. When the X-ray generating apparatus 101 emits X-rays while an object 1101 is arranged between the X-ray generating apparatus 101 and the X-ray imaging apparatus 104, an X-ray image of the object 1101 is obtained. The phosphor 105 for converting X-rays into visible light is arranged in the X-ray imaging apparatus 104, and absorption of the X-rays by the phosphor 105 influences the accuracy of energy subtraction processing. When N₀(E) represents a spectrum at energy E of the X-rays exposed from the X-ray generating apparatus 101, μ_{C}(E) represents a linear attenuation coefficient of the phosphor at the energy E of the phosphor 105, d_{C} represents a thickness, and P_{C} represents a filling rate, equation (15) below holds.

N(E)=N₀(E)[1−exp{−μ_{C}(E)d_{C}P_{C}}] (15)

In equation (15), an X-ray spectrum N(E) is a spectrum considering the X-ray absorption of the phosphor 105, and is obtained from the X-ray spectrum N₀(E) and parameters associated with the X-ray absorption of the phosphor 105. The X-ray spectrum N₀(E) of the X-rays emitted from the X-ray generating apparatus 101 is obtained by simulation or actual measurement. The linear attenuation coefficient μ_{C}(E) at the energy E of the phosphor 105, the thickness d_{C}, and the filling rate P_{C} are obtained from design values. A linear attenuation coefficient μ(E) of an arbitrary material at the energy E is obtained from a database of NIST or the like. Therefore, it is possible to calculate a thickness D of an arbitrary material and an attenuation rate I/I₀ for the X-ray spectrum N(E). Note that with respect to equation (15), only the linear attenuation coefficient is used for the sake of simplicity. However, in actual calculation, a more accurate result is obtained by considering an energy absorption coefficient. Not all the X-rays (X-rays indicated by equation (15)) that have not passed through the phosphor 105 are converted into a sensor output. Therefore, it is expected to improve the accuracy by considering the X-rays that have not been converted into the sensor output, that is, the energy absorption coefficient.

In FIG. 11, an arrangement 11b is an arrangement for obtaining a correction coefficient according to the third embodiment. A calibration sample 1102 is arranged between the X-ray generating apparatus 101 and the X-ray imaging apparatus 104. At this time, if the sample 1102 whose linear attenuation coefficient of X-rays, density, and thickness are already known is used, the calculated value of the attenuation rate I/I₀ of the sample 1102 is obtained from equation (3). The actually measured value of the attenuation rate I/I₀ of the sample 1102 is obtained by actually performing X-ray imaging with the arrangement 11b and obtaining an X-ray image.

In FIG. 11, 11c shows an example of actual measurement of the attenuation rate I/I₀. An obtained X-ray image 1121 is two-dimensional information of X columns×Y rows corresponding to the number of pixels 20. A region 1122 in the X-ray image 1121 is part of a region 1124 where X-rays passes through the sample 1102 to enter the X-ray imaging apparatus 104, and a region 1123 is part of a region 1125 where X-rays enters the X-ray imaging apparatus 104 without passing through the sample 1102. At this time, the actually measured value of the attenuation rate I/I₀ is obtained by obtaining an output average value I of the pixels 20 in the region 1122 and an output average value Jo of the pixels 20 in the region 1123. Note that before the attenuation rate I/I₀ is obtained, the offset correction, color correction, and dose dependence correction described in the first embodiment and the thickness dependence correction and energy dependence correction described in the second embodiment may be performed.

The calculated value and actually measured value of the attenuation rate I/I₀ obtained for the sample 1102 generally do not match each other. This is because the calculated value of the attenuation rate includes an error factor caused by the individual variation of the phosphor 105. To cope with this, such a linear attenuation coefficient μ_{C}′(E) of the phosphor 105, a thickness d_{C}′, and a filling rate P_{C}′ that the calculated value and actually measured value of the attenuation rate I/I₀ of the sample 1102 match each other are obtained. The linear attenuation coefficient μ_{C}′(E), the thickness d_{C}′, and the filling rate P_{C}′ may be obtained based on the difference between the calculated value and actually measured value of the attenuation rate I/I₀ of the sample 1102 or obtained by making the calculated value converge to the actually measured value by an iterative method.

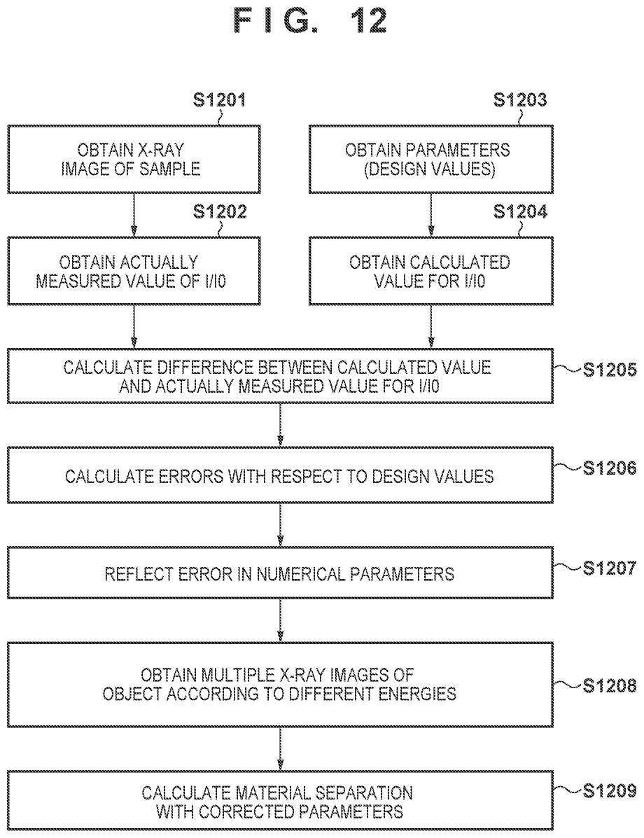

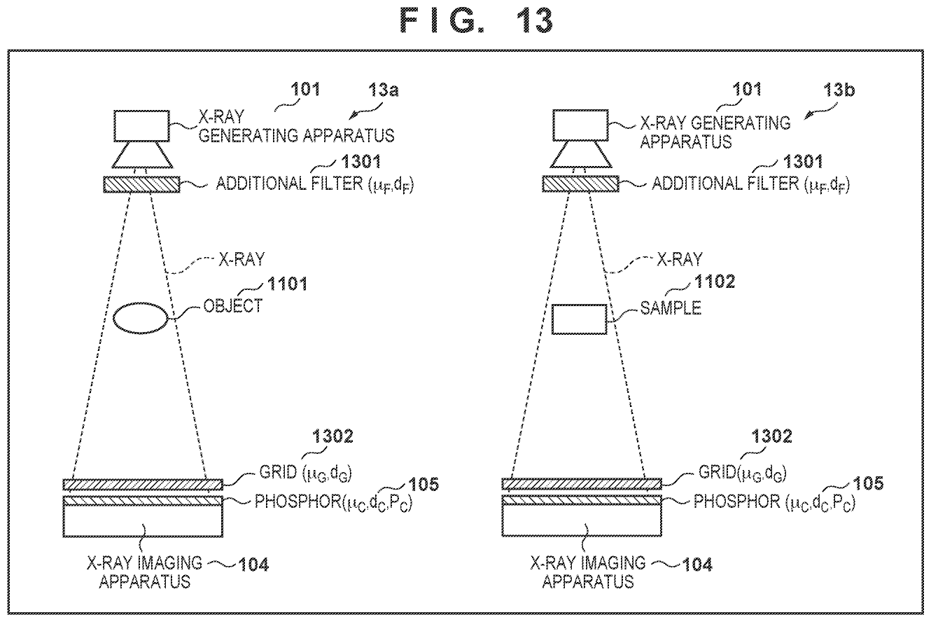

FIG. 12 shows a flowchart of correction processing according to the third embodiment. In step S1201, an obtaining unit 131 obtains, as an X-ray image of the sample 1102, the X-ray image 1121 shown in 11c of FIG. 11. In step S1202, a correcting unit 132 obtains the attenuation rate (I/I₀) using the pixel values of the regions 1122 and 1123 of the X-ray image 1121, and holds it as an actually measured value. On the other hand, in step S1203, the correcting unit 132 obtains the design values of the parameters (linear attenuation coefficient μ_{C}(E), thickness d_{C}, and filling rate P_{C}) used in equation (15). Then, in step S1204, the correcting unit 132 calculates the attenuation rate (I/I₀) using the parameters obtained in step S1203 and equation (15).

In step S1205, the correcting unit 132 calculates the difference between the actually measured value of the attenuation rate obtained in step S1202 and the calculated value of the attenuation rate obtained in step S1204. Then, in step S1206, the correcting unit 132 calculates errors with respect to the design values of the parameters of a target material (in this example, the phosphor 105) based on the difference calculated in step S1205. In step S1207, the correcting unit 132 obtains the corrected linear attenuation coefficient μ_{C}′(E), thickness d_{C}′, and filling rate P_{C}′ by reflecting the errors calculated in step S1206 on the parameters of the phosphor 105. These corrected parameters of the phosphor 105 are obtained by correcting the individual variation with respect to the design values. Therefore, these corrected parameters are substituted into equation (15), thereby obtaining the spectrum N(E) by correcting the individual variation of the phosphor 105. When equations (4) are solved using the thus obtained spectrum N(E), it is expected that the calculated estimated values of a bone thickness B and a soft tissue thickness S become close to true values.

In step S1208, the obtaining unit 131 obtains a plurality of X-ray images by capturing an object with a plurality of different X-ray energies, and the correcting unit 132 performs, for example, the processing described with reference to FIG. 7A or 7B to obtain a plurality of attenuation rate images corresponding to the plurality of different X-ray energies. In step S1209, the signal processing unit 133 generates the corrected X-ray spectrum N(E) using the corrected parameters generated in step S1207 and equation (15), and performs energy subtraction processing using the corrected X-ray spectrum N(E). Thus, material decomposition calculation is performed by the energy subtraction processing using the corrected X-ray spectrum, thereby obtaining, for example, bone and soft tissue thickness images.

Note that a plurality of data of the actually measured values (and calculated values) of the attenuation rate I/I₀ may be obtained by changing a pattern, as needed. As examples of changing the pattern, the energy of X-rays emitted by the X-ray generating apparatus 101 is changed, the material of the sample 1102 is changed, and the thickness of the sample 1102 is changed. The third embodiment has explained the case in which the three parameters (μ_{C}(E), d_{C}, and P_{C}) associated with the X-ray absorption of the phosphor 105 are corrected but the present invention is not limited to this. For example, a parameter as a correction target may include one or two of the three parameters, or may be arbitrarily selectable by the user. For example, only the thickness (d_{C}) of the phosphor may be corrected. As the number of parameters as correction targets increases, the necessary number of actually measured values (and calculated values) of the attenuation rate I/I₀ increases. Therefore, a parameter whose design variation of the material is small and which has a small influence on the attenuation rate may be excluded from the correction target. Other parameters such as a mass attenuation coefficient and a density that influence attenuation of X-rays may be corrected.

Note that when a spectrum difference Δμ_{C}(E), a thickness difference Δd_{C}, and a filling rate difference ΔP_{C} between the design values (μ_{C}(E), d_{C}, and P_{C}) and the corrected values (μ_{C}′(E), d_{C}′, and P_{C}′) are set as correction values, the absolute values (correction ranges) of the correction values are preferably limited to the design variation ranges. This is to prevent the correction values from taking unrealistic values.

In the third embodiment, when obtaining the actually measured value of the attenuation rate I/I₀ of the sample 1102, a method of providing the region 1124 with the sample and the region 1125 without the sample in the X-ray image 1121 and calculating an average value in each region is used. The present invention, however, is not limited to this. For example, imaging may be performed a plurality of times so as to obtain an image with the sample and an image without the sample. At this time, correction values may be obtained for each pixel 20, and the above-described parameters may be corrected for each pixel. At this time, the correction values are two-dimensional information of X columns×Y rows, and in-plane variations of the target substance (in this example, the phosphor 105) can also be corrected.

### Fourth Embodiment

The third embodiment has explained the arrangement considering X-ray absorption by the phosphor. The fourth embodiment will describe a case in which in addition to an object, an additional filter and/or a scattered ray removal grid exists, as an arrangement for absorbing X-rays, between an X-ray generating apparatus 101 and an X-ray imaging apparatus 104 (phosphor 105). The block diagram of an imaging system and the equivalent circuit diagram of a pixel 20 according to the fourth embodiment are the same as in the first embodiment (FIGS. 1 and 2).

In FIG. 13, 13a shows an example (arrangement 13a) of an X-ray imaging arrangement according to the fourth embodiment. An object 1101 is arranged between the X-ray generating apparatus 101 and the X-ray imaging apparatus 104. Furthermore, an additional filter 1301 is attached to the X-ray generating apparatus 101, and a scattered ray removal grid 1302 is attached to the X-ray imaging apparatus 104. A metal filter of Al or Cu is preferably used as the additional filter 1301, and cuts a low energy X-ray region. The grid 1302 removes scattered rays that can be generated by the object 1101. Let N₀(E) be an X-ray spectrum exposed from the X-ray generating apparatus 101, and N′(E) be an X-ray spectrum after passing through the additional filter 1301 and the grid 1302. When d_{F} represents the thickness of the filter, d_{G} represents the thickness of the grid, μ_{F}(E) represents the linear attenuation coefficient of a bone at energy E, and μ_{G}(E) represents the linear attenuation coefficient of the grid at the energy E, equation (16) below holds with respect to X-rays reaching the phosphor 105.

N′(E)=N₀(E)exp{−μ_{F}(E)d_{F}−μ_{G}(E)d_{G}} (16)

Furthermore, when d_{C} represents the thickness of the phosphor 105, μ_{C}(E) represents the linear attenuation coefficient of the phosphor at the energy E, and P_{C} represents a filling rate, equation (17) below holds. An X-ray spectrum N(E) of equation (17) is a spectrum considering X-ray absorption of the additional filter 1301, the grid 1302, and the phosphor 105.

N(E)=N′(E)[1−exp{−μ_{C}(E)d_{C}P_{C}}] (17)

By solving nonlinear simultaneous equations (4), similar to the third embodiment, using the X-ray spectrum N(E) obtained by equations (16) and (17), a bone thickness B and a soft tissue thickness S are obtained. The estimated values of the bone and soft tissue thicknesses obtained by solving equations (4) in the arrangement 13a shown in FIG. 13 have errors with respect to true values. This is because the individual variations of the additional filter 1301 and the grid 1302 are included in addition to the individual variation of the phosphor 105 described in the third embodiment.

In FIG. 13, 13b shows a correction coefficient obtaining arrangement according to the fourth embodiment. A sample 1102 is arranged between the X-ray generating apparatus 101 and the X-ray imaging apparatus 104. A material whose attenuation coefficient of X-rays, density, and thickness are already known is used for the sample 1102. The procedure of correction processing is the same as in the third embodiment (FIG. 12). A correcting unit 132 obtains a calculated value of an attenuation rate I/I₀ of the sample 1102 based on equations (4), (16), and (17) using the design values (parameters) of the additional filter 1301, the grid 1302, the phosphor 105, and the sample 1102 (S1203 and S1204). The correcting unit 132 obtains an X-ray image by performing X-ray imaging using the arrangement 13b shown in FIG. 13, thereby obtaining the actually measured value of the attenuation rate I/I₀ of the sample 1102 (S1201 and S1202).

The correcting unit 132 obtains corrected parameters (S1205 to S1207) by the same method as in the third embodiment. The corrected parameters are, for example, a linear attenuation coefficient μ_{C}′(E) of the phosphor 105, a thickness d_{C}′, a filling rate P_{C}′, a linear attenuation coefficient μ_{F}′(E) of the additional filter 1301, a thickness d_{F}′, a linear attenuation coefficient μ_{G}′(E) of the grid 1302, and a thickness d_{G}. The signal processing unit 133 solves equations (4) using the spectrum N(E) obtained by substituting these corrected parameters into equations (16) and (17), thereby obtaining the estimated values of the bone thickness B and the soft tissue thickness S (S1208 and S1209). As described above, by performing energy subtraction processing using the corrected X-ray spectrum N(E), it is expected that the estimated values of the bone thickness B and the soft tissue thickness S become close to the true values.

The fourth embodiment has explained the case in which the additional filter and the grid are attached as X-ray imaging accessories but the present invention is not limited to this. For example, only one of the additional filter and the grid may be attached or another accessory may be added. Furthermore, if a material that can absorb X-rays exists between the X-ray generating apparatus 101 and the phosphor 105 in addition to the object, the material may be set as a correction target. An example of the material is an accessory of the X-ray imaging apparatus.

### Fifth Embodiment

In each of the third and fourth embodiments, the parameters associated with X-ray absorption of an arrangement (substance) other than the object are calibrated based on the attenuation rate HO as a sensor output, thereby correcting the X-ray spectrum. In the fifth embodiment, the above calibration processing is performed based on the processing result of energy subtraction processing using a plurality of attenuation rate images corresponding to a plurality of X-ray energies. The block diagram of an imaging system and the equivalent circuit diagram of a pixel 20 according to the fifth embodiment are the same as in the first embodiment (FIGS. 1 and 2).

An arrangement for obtaining a correction coefficient according to the fifth embodiment is the same as in the third embodiment (arrangement 11b). That is, a calibration sample 1102 is arranged between an X-ray generating apparatus 101 and an X-ray imaging apparatus 104. The sample 1102 is formed from only portions of a bone and a soft tissue (or materials having X-ray attenuation coefficients equal to those of the portions), and the thickness of each portion is already known. X-ray imaging is performed with the arrangement 11b shown in FIG. 11 to obtain an X-ray image, thereby obtaining the actually measured value of an attenuation rate I/I₀ of the calibration sample 1102. In this state, an attenuation rate image (high energy image) at high energy and an attenuation rate image (low energy image) at low energy are obtained, thereby making it possible to obtain a bone thickness image B and a soft tissue thickness image S in the sample 1102. The thus obtained images B and S do not match the actual thicknesses of the respective portions of the sample 1102, respectively, due to errors of X-ray spectra N_{H}(E) and N_{L}(E). Therefore, it is necessary to calibrate the X-ray spectra. In the calibration method of each of the third and fourth embodiments, the X-ray spectra are modified so that the actually measured value of the attenuation rate I/I₀ matches the value calculated from the X-ray spectra. However, even if the error of the attenuation rate becomes small, thickness errors (errors of the result of energy subtraction processing) may not become small.

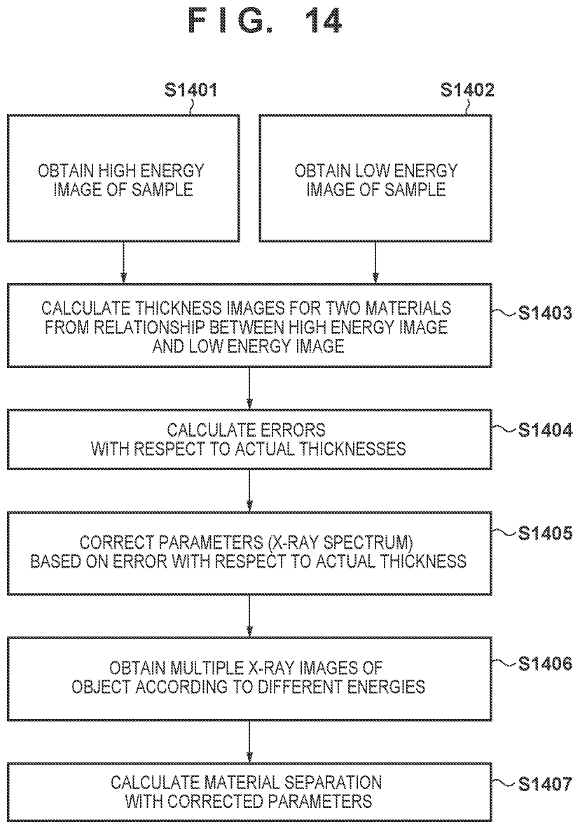

In the fifth embodiment, the X-ray spectra are calibrated by processing shown in FIG. 14. FIG. 14 is a flowchart illustrating correction processing according to the fifth embodiment. In steps S1401 and S1402, an obtaining unit 131 obtains a plurality of X-ray images by performing X-ray imaging with a plurality of different X-ray energies in the arrangement 11b of FIG. 11. A correcting unit 132 generates an attenuation rate image (high energy image) at high energy and an attenuation rate image (low energy image) at low energy from the plurality of X-ray images corresponding to the plurality of different X-ray energies obtained by the obtaining unit 131. In step S1403, the signal processing unit 133 performs energy subtraction processing using the high energy image and the low energy image obtained in steps S1401 and S1402, respectively, and calculates the bone thickness B and the soft tissue thickness S of the sample 1102. In step S1404, the correcting unit 132 calculates errors between the calculated values of the thicknesses B and S and the actually measured values of the thicknesses B and S of the sample 1102. Then, in step S1405, the X-ray spectra N_{H}(E) and N_{L}(E) are corrected so that the errors calculated in step S1404 become small.

Next, in step S1406, the obtaining unit 131 and the correcting unit 132 obtain a plurality of X-ray images corresponding to a plurality of different energies by capturing X-ray images of an object 1101, and obtain a plurality of attenuation rate images corresponding to the plurality of different energies based on the obtained X-ray images. In step S1407, the signal processing unit 133 performs material decomposition using the corrected spectra N_{H}(E) and N_{L}(E). It is considered that a decomposition result closer to a true value is obtained with respect to the object 1101 by the above processing.

An example of the method of correcting the X-ray spectra in step S1405 will be described. Assume that an unexpected substance having an attenuation coefficient μ(E) and a thickness d is arranged between the X-ray generating apparatus 101 as an X-ray source and the X-ray imaging apparatus 104 as a sensor. In this case, when N(E) represents an original X-ray spectrum, equation (18) below holds with respect to a corrected X-ray spectrum N_{C}(E).

N_{C}(E)=N(E)exp(−μ(E)d) (18)

The thicknesses B and S of the sample 1102 are calculated using N_{C}(E), and such μ(E) and d that errors between the calculated values and actual values of the thicknesses become small are obtained. In the fifth embodiment, the spectra are calibrated based on a physical model in which a material whose attenuation coefficient and thickness are unknown is arranged between the X-ray source and the sensor. However, the present invention is not limited to this. The spectra may randomly be modified, or it may be assumed that a specific material whose effective atomic number and area density are determined may be sandwiched.

An optimization procedure in spectrum calibration will be described. First, the value of a material of an effective atomic number Z and an area density D is randomly decided. At this time, it is preferable to limit Z and D to realistic values. Next, high and low energy spectra are modified based on the decided values Z and D according to equation (17). Then, Z and D are calculated based on the modified spectra, and an RMSE (Root Mean Square Error) with respect to the actual measurement is obtained. The above spectrum modification processing is repeated a predetermined number of times, and a spectrum having the smallest RMSE is obtained. The above processing is repeated a predetermined number of times to obtain a plurality of spectra, and these spectra are averaged. The averaged spectrum is set as a spectrum before calibration, and the same procedure is repeated a predetermined number of times. The above processing optimizes the spectrum. Note that the present invention is not limited to this. For example, the atomic number Z and the area density D need not always take realistic values, and the spectrum modification processing need not always be repeated.

The spectrum N(E) according to the fifth embodiment indicates a spectrum considering X-ray absorption of the phosphor 105, as indicated by equation (15), and a value obtained by modifying, based on the arrangement when performing imaging, the X-ray spectrum generated from the X-ray generating apparatus 101 is used as the spectrum N(E). This embodiment has explained the case in which the spectrum N(E) is modified but the present invention is not limited to this. Parameters used for spectrum modification may be corrected. For example, a thickness d_{C} and a filling rate P_{C} of the phosphor may be corrected.

### Sixth Embodiment

In the fifth embodiment, a sensor output generated when one X-ray photon is absorbed by the phosphor is proportional to the energy E of the X-ray photon. That is, it is assumed that the conversion efficiency from X-rays into a sensor output does not depend on energy. The sixth embodiment will describe correction when considering the energy dependence of the conversion efficiency of a sensor. The block diagram of an imaging system and the equivalent circuit diagram of a pixel 20 according to the sixth embodiment are the same as in the first embodiment (FIGS. 1 and 2).

When E represents the energy of an X-ray photon, N(E) represents an X-ray spectrum, C(E) represents the conversion efficiency of a sensor output, D represents the thickness of an arbitrary material, μ(E) represents the linear attenuation coefficient of the arbitrary material, and I/I₀ represents an attenuation rate, equation (19) below holds.

$$I / {I}_{0} = \frac{{\int }_{0}^{∞} C ( E ) N ( E ) exp \{ - μ ( E ) D \} E d E}{{\int }_{0}^{∞} C ( E ) N ( E ) E d E} \qquad ( 19 )$$

Assuming that N₁(E)=C(E)N(E) in equation (19), the same equation as equation (3) is obtained. Therefore, thicknesses B and S of a sample 1102 used in the fifth embodiment are obtained by performing the same calculation processing (energy subtraction processing) as in the first embodiment for a high energy image and a low energy image obtained by performing X-ray imaging of the sample 1102. The conversion efficiency C(E) can be calibrated based on errors between the thicknesses B and S of the sample 1102 obtained by the calculation processing and actual thicknesses B and S of the sample 1102.

A method of calibrating C(E) will be described. The conversion efficiency C(E) before calibration can be regarded as C(E)=1. This is calibrated by fitting using an appropriate function. For example, C(E)=aE+b is assumed. The thicknesses B and S are calculated by randomly deciding variables a and b, and RMSEs (Root Mean Square Errors) with respect to the actual thicknesses B and S are obtained. The above processing is repeated a predetermined number of times to obtain a and b that give the smallest RMSEs, thereby obtaining a calibrated conversion efficiency Cc(E). The signal processing unit 133 captures an X-ray image of the object 1101, and performs material decomposition using the calibrated conversion efficiency Cc(E). Thus, by performing energy subtraction processing using Cc(E)N(E) as a calibrated X-ray spectrum, it is expected to obtain a decomposition result (thicknesses B and S) closer to a true value with respect to the object 1101.

Note that in the sixth embodiment, calibration is performed based on the RMSEs of B and S by assuming C(E) as a linear function. The present invention, however, is not limited to this. For example, the function C(E) may be a quadratic function or a linear combination of natural logarithms or the like. Alternatively, C(E) may be calibrated not based on the RMSEs of B and S but based on the differences or relative errors between the actually measured values and the calculated values. The sixth embodiment has explained the case in which C(E) is corrected based on the errors between the calculated values and actually measured values of B and S but the present invention is not limited to this. For example, correction may be performed based on the thickness of only B or N(E) may be corrected at the same time in addition to C(E).

Note that in each of the first to sixth embodiments, the bone thickness B and the soft tissue thickness S are calculated by the energy subtraction processing but the present invention is not limited to this. For example, a water thickness W and a contrast agent thickness I may be calculated. That is, the present invention can be applied to decomposition into the thicknesses of arbitrary two kinds of materials. An arrangement for calculating an image of the effective atomic number Z and an image of the area density D by the energy subtraction processing from the attenuation rate image L at low energy and the attenuation rate image H at high energy may be used. The effective atomic number Z indicates the equivalent atomic number of a mixture, and the area density D is the product of the density [g/cm³] of the object and the thickness [cm] of the object. Furthermore, the image processing unit 134 may generate a virtual monochromatic X-ray image using the effective atomic number Z and the area density D. The image processing unit 134 may generate a composite X-ray image by compositing a plurality of virtual monochromatic X-ray images generated by the plurality of energies E_{V}. The composite X-ray image indicates an image assumed to be obtained when X-rays of an arbitrary spectrum are emitted.

In each of the above-described first to sixth embodiments, an indirect X-ray sensor using a phosphor is used as the X-ray imaging apparatus 104. The present invention, however, is not limited to this. For example, a direct X-ray sensor using a direct conversion material such as CdTe may be used. That is, the X-ray sensor may be either an indirect or direct X-ray sensor. In each of the first and second embodiments, for example, the tube voltage of the X-ray generating apparatus 101 is changed in the operation shown in FIG. 4. However, the present invention is not limited to this. The energy of X-rays exposed to the X-ray imaging apparatus 104 may be changed by temporally switching the filter of the X-ray generating apparatus 101. That is, the method of changing the energy of X-rays exposed to the X-ray imaging apparatus 104 is not particularly limited. The second embodiment assumes that execution of dose dependence correction according to the first embodiment but the present invention is not limited to this. Only the attenuation rate correction processing described in the second embodiment may be performed. An arrangement for executing only one of correction of thickness dependence and correction of energy dependence may be adopted.

In each of the first to sixth embodiments, two X-ray energies are used but the present invention is not limited to this. Even if three or more X-ray energies are used, for example, an arrangement for performing correction processes in the order of offset correction→dose dependence correction→color correction→gain correction→attenuation rate correction, as shown in FIG. 10, can be applied. That is, the processes of the above embodiments are also applicable to X-ray images obtained by three or more X-ray energies.

Furthermore, in FIG. 10, the correction processing order shown in FIG. 7B is applied. However, the correction processing order (offset correction→color correction→dose dependence correction→gain correction→attenuation rate correction) shown in FIG. 7A may be applied. In each of the first to sixth embodiments, images of different energies are obtained by changing the X-ray energy but the present invention is not limited to this. For example, a stacked arrangement for obtaining images of different energies from a front two-dimensional detector and a rear two-dimensional detector with respect to the incident direction of X-rays by overlaying a plurality of phosphors 105 and a plurality of two-dimensional detectors 106 may be used. In this case, color correction by the correcting unit 132 is unnecessary.

In each of the first to sixth embodiments, the imaging control apparatus 103 of the X-ray imaging system is used to perform the energy subtraction processing. However, the present invention is not limited to this. For example, images obtained by the imaging control apparatus 103 may be transferred to another computer and then the energy subtraction processing may be performed. For example, obtained images may be transferred to another personal computer via a medical PACS, may undergo the energy subtraction processing, and may then be displayed. That is, the apparatus for performing the correction processing described in each of the above embodiments need not be a set with the imaging apparatus (may be an image viewer).

According to the above-described embodiments, an error occurring when generating a material characteristic image by energy subtraction can be reduced.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a generating unit configured to generate a plurality of attenuation rate images using a plurality of radiation images corresponding to a plurality of different radiation energies obtained by irradiating an object with radiation;
a correcting unit configured to correct the radiation images or the attenuation rate images so as to reduce an error of an attenuation rate caused depending on at least one of a dose of the radiation, a thickness of the object, and energy of the radiation; and
a processing unit configured to generate a material characteristic image by energy subtraction processing using the plurality of attenuation rate images after the correction by the correcting unit.

2. The apparatus according to claim 1, wherein the correcting unit corrects a pixel value of the radiation image using a correction coefficient so that the pixel value and the dose are proportional to each other.

3. The apparatus according to claim 2, wherein the correcting unit corrects, using the correction coefficient, the pixel value of the radiation image after executing offset correction.

4. The apparatus according to claim 2, wherein
the plurality of radiation images are obtained by performing a sample and hold operation a plurality of times during exposure of radiation of one shot, and
the correcting unit includes color correction for obtaining an image corresponding to radiation during a specific period divided by the sample and hold operation by subtraction of the plurality of radiation images, and performs correction for an image after execution of the color correction using the correction coefficient.

5. The apparatus according to claim 2, wherein when x represents a pixel value and g(x) represents the correction coefficient, the correcting unit obtains a corrected pixel value f(x) using f(x)=x×g(x), and the correction coefficient g(x) is expressed by a quadratic function.

6. The apparatus according to claim 2, wherein the correcting unit performs the correction with reference to a table that stores a relationship between a pixel value x and a corrected pixel value f(x).

7. The apparatus according to claim 1, wherein the correcting unit corrects a pixel value of the plurality of attenuation rate images so as to reduce one of the error depending on the thickness and the error depending on the energy of the radiation.

8. The apparatus according to claim 7, wherein the correcting unit corrects the pixel value of the plurality of attenuation rate images so that a change of the attenuation rate with respect to the thickness of the object matches a predetermined curve or straight line or a change of the attenuation rate with respect to average energy of the radiation matches a predetermined curve or straight line.

9. An image processing apparatus comprising:
a correcting unit configured to correct, based on a difference between a pixel value obtained by calculation and a pixel value obtained by actual measurement, a radiation spectrum to be used for energy subtraction processing;
a generating unit configured to generate a plurality of attenuation rate images using a plurality of radiation images corresponding to a plurality of different radiation energies obtained by irradiating an object with radiation; and
a processing unit configured to generate a material characteristic image by energy subtraction processing using the plurality of attenuation rate images and the radiation spectrum corrected by the correcting unit.

10. The apparatus according to claim 9, wherein the correcting unit corrects the radiation spectrum so that at least one of an attenuation rate measured from the radiation images obtained with respect to a sample of a plurality of thicknesses and an attenuation rate measured from radiation images of the sample obtained by a plurality of radiation energies matches an attenuation rate of the sample calculated using the radiation spectrum.

11. The apparatus according to claim 9, wherein the correcting unit corrects the radiation spectrum based on an attenuation rate calculated using the radiation spectrum and an attenuation rate obtained by actual measurement or based on a material characteristic calculated by the energy subtraction processing and a material characteristic obtained by actual measurement.

12. The apparatus according to claim 11, wherein the correcting unit corrects a radiation spectrum of each of the plurality of radiation energies based on radiation absorption by an arrangement other than the object, corrects the radiation spectrum so that a thickness of a sample whose thickness calculated by the energy subtraction processing is already known matches an actual thickness of the sample, or calculates conversion efficiency of a sensor of a radiation imaging apparatus and corrects the radiation spectrum using the conversion efficiency so that the calculated attenuation rate and the attenuation rate obtained by the actual measurement match each other.

13. The apparatus according to claim 12, wherein the correcting unit corrects a parameter for calculating radiation absorption by the arrangement so that an attenuation rate obtained by irradiating, with radiation, a sample whose linear attenuation coefficient and thickness are already known and performing imaging matches an attenuation rate calculated using the radiation spectrum and the linear attenuation coefficient and the thickness of the sample, or corrects the radiation spectrum by calculating an attenuation coefficient and a thickness of a virtual substance arranged between a radiation generating apparatus and the radiation imaging apparatus and correcting the attenuation coefficient and the thickness of the virtual substance so that the calculated thickness matches the actual thickness.

14. The apparatus according to claim 13, wherein the parameter for calculating the radiation absorption by the arrangement includes at least one of a linear attenuation coefficient, a thickness, and a filling rate.

15. The apparatus according to claim 13, further comprising a selecting unit configured to cause a user to select a correction target parameter.

16. The apparatus according to claim 12, wherein the arrangement includes a phosphor configured to convert radiation energy into visible light, or the arrangement includes an additional filter and/or a grid arranged between a radiation generating apparatus and the radiation imaging apparatus.

17. An image processing apparatus comprising a processing unit configured to perform energy subtraction processing (a) using a radiation spectrum obtained by performing correction based on a plurality of attenuation rates corresponding to a plurality of different radiation energies obtained by irradiating an object with radiation and a difference between a pixel value obtained by calculation and a pixel value obtained by actual measurement, or (b) using at least one attenuation rate obtained by performing correction so as to reduce an error of at least one attenuation rate caused depending on at least one of a dose of the radiation, a thickness of the object, and energy of the radiation.

18. An image processing method comprising performing energy subtraction processing (a) using a radiation spectrum obtained by performing correction based on a plurality of attenuation rates corresponding to a plurality of different radiation energies obtained by irradiating an object with radiation and a difference between a pixel value obtained by calculation and a pixel value obtained by actual measurement, or (b) using at least one attenuation rate obtained by performing correction so as to reduce an error of at least one attenuation rate caused depending on at least one of a dose of the radiation, a thickness of the object, and energy of the radiation.

19. A non-transitory computer-readable storage medium storing a program for causing a computer to execute the method according to claim 18.
